(19) United States
(12) Patent Application Publication
(10) Pub. No.: US 2008/0204043 A1
(43) Pub. Date: Aug. 28, 2008

(54) Real-Time Assessment of Biomarkers for Disease

(75) Inventors: Kevin K.W. Wang, Gainesville, FL (US); Andrew K. Ottens, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: University of Florida Research Foundation, Inc., Gainesville, FL (US)

(21) Appl. No.: 11/666,397

(22) PCT Filed: Oct. 27, 2005
(86) PCT No.: PCT/US05/39037

§ 371 Date: Mar. 17, 2008

Related U.S. Application Data
(60) Provisional application No. 60/622,381, filed on Oct. 27, 2004.

(51) Int. Cl. G01N 33/53; B01J 19/00; G01N 33/487

(52) U.S. Cl. 324/633; 422/68.1; 436/501

(57) ABSTRACT

A system and methods, thereof, monitor biomarkers from a patient suffering from neural injury in real-time. The system comprises a quartz crystal microbalance wherein a capture molecule specific for certain biomarkers is diagnostic of the type of neural injury, location of neural injury and the degree of severity of neural injury. The system, in particular, provides real-time continuous monitoring of a patient.

### FIELD OF THE INVENTION

Another major drawback of whole animal experiments in the number of variables which cannot be controlled and are difficult to assess. For example, if a device and methods for the real-time monitoring of a patient suffering from neural injury and/or disorders. In particular, the device monitors in real-time, biomarker levels in biological fluids such as CSF, blood etc. in acute human disease states such as traumatic brain injury (TBI), cerebral vascular accidents (CVA), myocardial infarction (MI), and other organ failures in real-time.

### BACKGROUND OF THE INVENTION

The incidence of traumatic brain injury (TBI) in the United States is conservatively estimated to be more than 2 million persons annually with approximately 500,000 hospitalizations. Of these, about 70,000 to 90,000 head injury survivors are permanently disabled. The annual economic cost to society for care of head-injured patients is estimated at $25 billion. These figures are for the civilian population only and the incidence is much greater when combat casualties are included. In modern warfare (1993-2000), TBI is the leading cause of death (53%) among wounded who have reached medical care facilities.

Assessment of pathology and neurological impairment immediately after TBI is crucial for determination of appropriate clinical management and for predicting long-term outcome. The outcome measures most often used in head injuries are the Glasgow Coma Scale (GCS), the Glasgow Outcome Scale (GOS), computed tomography, and magnetic resonance imaging (MRI) to detect intracranial pathology. However, despite dramatically improved emergency triage systems based on these outcome measures, most TBI suffer long term impairment and a large number of TBI survivors are severely affected despite predictions of “good recovery” on the GOS. In addition, CT and MRI are expensive and cannot be rapidly employed in an emergency room environment. Moreover, in austere medical environments associated with combat, accurate diagnosis of TBI would be an essential prerequisite for appropriate triage of casualties.

It is very desirable to rapidly detect and quantify one or more molecular structures in a sample from such patients. The molecular structures typically comprise without limitation, agonists and antagonists for cell membrane receptors, toxins, venoms, oligosaccharides, proteins, bacteria and monoclonal antibodies. Presently, there are fundamental constraints to the analysis and diagnosis of patients suffering from neural injury and/or neural disorders, e.g., limited sample, time, or often both. There is therefore a need in the art to provide a system for the detection and diagnosis of injuries affecting the neural system and such a need must balance between accuracy, speed, and sensitivity.

### SUMMARY

The present invention generally relates to test systems and methods for characterizing neural injury and/or disorder in a mammal in real-time, and particularly a human patient. In general, the test systems and methods detect at least one neural biomarker from a patient sample and relate the neural biomarker to the presence and preferably the stage of the neural injury and/or disorder in the mammal. The present invention has several important applications including use in the diagnosis and/or treatment of neural injury and/or disorders in human patients.

More particularly, neural biomarkers can be used to detect in real-time and preferably, stage of neural injury and/or disorders using a system comprising a quartz crystal microbalance. Sometimes the neural biomarker molecules will be referred to herein as neural “biomarkers” or a similar term to denote a physiological origin for these molecules. Preferred use of the invention generally involves detection and quantitation of biomarkers that have been found to be indicative of the presence and certain stage of neural injury and/or disorders, in real-time. For example, the extent of neural injury and/or the presence and stage of neural disorders can be determined with high sensitivity and selectivity by detecting and quantifying the biomarkers in real-time. Preferred use of the invention is via a bedside biosensor monitor, thereby facilitating testing of sick, infirm or very young patients including children and infants. The test systems and methods described herein are well-suited for safe and reliable handling of large numbers of samples.

The present invention has additional important uses and advantages. For example, it can be used to provide continuous and reliable information as to the extent of neural injury and/or disorder of an injured patient and/or one suffering from or susceptible to a neural disorder and in real-time. Opportunities for early medical intervention are therefore increased. The invention can be used to monitor neural injury of accident victims at the scene of an accident, thereby, providing the attendant physician or medic with instantaneous and continuous data in such patients. Alternatively, the invention can be used to evaluate neural injury as part of a diagnosis of a neural disorder.

In a preferred embodiment, the invention provides a system with a flow-through biomonitor comprising a surface substrate for adsorbing a first molecule that specifically binds a second molecule; a means of detection of such binding; a means of evaluating and correlating the binding of the molecules to extent of neural injury and/or neural disorder.

In another preferred embodiment, the biomonitor is sensitive to minute differences in measurement of mass. Preferably, the biomonitor comprises a quartz crystal microbalance (QCM) as a surface substrate for adsorbing a first molecule. The principal components of a QCM system include a QCM sensor, an oscillator and control circuitry. In a QCM sensor there are typically two carefully matched quartz crystals aligned parallel to each other and separated by a small gap. Optionally, only one of the crystals, can be exposed to the sample. The difference in frequency between the two crystals is the beat frequency, which is a very sensitive indication of the mass being deposited on the sample exposed crystal surface. The beat frequency is proportional to the mass of the surface adsorbed first molecule and the second molecule which specifically binds to the adsorbed molecule which have accumulated on the sensing area. The accumulated mass is electronically recorded in a digital electronic counter.

The quartz crystal can be about 100 nm thick, 0.5 mm diameter and the surface thickness ranges from about 1 nm to about 1000 nm, a diameter from 0.1 to 25 mm.

In another preferred embodiment, the biomonitor comprises control circuits associated with QCM sensors. For example, controls include, but are not limited to an assembly of circuitry and sensors, such as, a QCM sensor signal conditioner, a QCM sensor temperature monitor, a thermal-electric heat pump controller and a microcontroller for data acquisition and data formatting. These elements have been collectively referred to as QCM controllers, controllers, or control circuits.

In another preferred embodiment, the QCM sensors comprise controllers to measure the sensor's resonant frequency over a wide range of impedance. Other examples of QCM sensors, include but are not limited to QCM sensors that are used to measure the mass of a substantial drop of liquid or particulate matter and are designed to correct for significant viscous damping losses. Preferably, the QCM sensor and controller are accessible; therefore, self-monitoring and wireless telemetry are not needed.

In another preferred embodiment, QCM sensors comprise controllers to measure, in addition to determining mass, the molecular species of the material deposited on the QCM sensor's quartz crystal.

In one aspect, the amount of each biomarker is measured in the subject sample and the ratio of the amounts between the markers is determined, such as, for example, any two or more biomarkers or combinations thereof, illustrated in Table 1, infra. Preferably, the amount of each biomarker in the subject sample and the ratio of the amounts between the biomarkers and compared to normal healthy individuals. The increase in ratio of amounts of biomarkers between healthy individuals and individuals suffering from injury is indicative of the injury magnitude, disorder progression as compared to clinically relevant data.

Preferably, biomarkers that are detected at different stages of injury and clinical disease are correlated to assess anatomical injury, type of cellular injury, subcellular localization of injury. Monitoring of which biomarkers are detected at which stage, degree of injury in disease or physical injury provides panels of biomarkers that provide specific information on mechanisms of injury, identify multiple subcellular sites of injury, identify multiple cell types involved in disease related injury and identify the anatomical location of injury. Examples of injury include but not limited to: subarachnoid hemorrhage, epilepsy, stroke, and other forms of brain injury.

In another aspect, preferably a single biomarker is used in combination with one or more biomarkers from normal, healthy individuals for diagnosing injury, location of injury and progression of disease and/or neural injury, more preferably a plurality of the markers are used in combination with one or more biomarkers from normal, healthy individuals for diagnosing injury, location of injury and progression of disease and/or neural injury. It is preferred that one or more protein biomarkers are used in comparing protein profiles from patients susceptible to, or suffering from disease and/or neural injury, with normal subjects.

In another preferred method, data is generated on immobilized subject samples on a QCM surface and detecting changes in molecular weight. Preferably, the QCM sensors comprise controllers to measure, in addition to determining mass, the molecular species of the material deposited on the QCM sensor's quartz crystal. The data are transformed into computer readable form; and executing an algorithm that classifies the data according to user input parameters, for detecting signals that represent markers present in injured and/or diseased patients and are lacking in non-injured and/or diseased subject controls.

In another preferred embodiment, the presence of certain biomarkers is indicative of the extent of CNS and/or brain injury. For example, detection of one or more dendritic damage markers, soma injury markers, demyelination markers, axonal injury markers would be indicative of CNS injury and the presence of one or more would be indicative of the extent of nerve injury. Examples of such biomarkers are shown in Table 1.

In another preferred embodiment, the presence of certain biomarkers is indicative of a neurological disorder, i.e. dendritic damage markers, soma injury markers, demyelination markers, axonal injury markers, synaptic terminal markers, post-synaptic markers.

Preferred methods for detection and diagnosis of CNS/PNS and/or brain injury comprise detecting at least one or more protein biomarkers in a subject sample, and; correlating the detection of one or more protein biomarkers with a diagnosis of CNS and/or brain injury, wherein the correlation takes into account the detection of one or more biomarker in each diagnosis, as compared to normal subjects, wherein the one or more protein markers are selected from: neural proteins, such as for example, axonal proteins—NF-200 (NF-H), NP-160 (NF-M), NF-68 (NF-L); amyloid precursor protein; dendritic proteins—alpha-tubulin (P02551), beta-tubulin (P0 4691), MAP-2A/B, MAP-2C, Tau, Dynamin-1 (P21575), Dynactin (Q13561), P24; somal proteins—UCH-L1 (Q00981), PEBP (P31044), NSE (P07323), Thy 1.1, Prion, Huntington, CK-BB (P07335); presynaptic proteins—synapsin-1, synapsin-2, alpha-synuclein (p37377), beta-synuclein (Q63754), GAP43, synaptophysin, synaptotagmin (P21707), syntaxin; post-synaptic proteins—PSD95, PSD93, NMDA-receptor (including all subtypes); demyelination biomarkers—myelin basic protein (MBP), myelin proteolipid protein; glial proteins—GFAP (P47819), protein disulfide isomerase (PDI-P04785); neurotransmitter biomarkers—cholinergic biomarkers: acetylcholine esterase, choline acetyltransferase; dopaminergic biomarkers—tyrosine hydroxylase (TH), phospho-TH, DARPP32; noradrenergic biomarkers—dopamine beta-hydroxylase (DbH); serotonergic biomarkers—tryptophan hydroxylase (TrH); glutamatergic biomarkers—glutaminase, glutamine synthetase; GABAergic biomarkers—GABA transaminase (4-aminobutyrate-2-ketoglutarate transaminase [GABAT]), glutamic acid decarboxylase (GAD25, 44, 65, 67); neurotransmitter receptors—beta-adrenoreceptor subtypes, (e.g. beta (2)), alpha-adrenoreceptor subtypes, (e.g. (alpha (2c)), GABA receptors (e.g. GABA(B)), metabotropic glutamate receptor (e.g. mGluR3), NMDA receptor subunits (e.g. NR1A2B), Glutamate receptor subunits (e.g. GluR4), 5-HT serotonin receptors (e.g. 5-HT(3)), dopamine receptors (e.g. D4), muscarinic Ach receptors (e.g. M1), nicotinic acetylcholine receptor (e.g. alpha-7); neurotransmitter transporters—norepinephrine transporter (NET), dopamine transporter (DAT), serotonin transporter (SERT), vesicular transporter proteins (VMAT1 and VMAT2), GABA transporter vesicular inhibitory amino acid transporter (VIAAT/VGAT), glutamate transporter (e.g. GLT1), vesicular acetylcholine transporter, choline transporter (e.g. CHT1); other protein biomarkers include, but not limited to vimentin (P31000), 14-3-3-epsilon (P42655), MMP2, MMP9.

Preferably, the biological sample is a fluid in communication with the nervous system of the subject prior to being isolated from the subject; for example, CSF or blood, and the agent can be an antibody, aptamer, or other molecule that specifically binds at least one or more of the neural proteins.

In another preferred embodiment, it is desirable to have a sensor-oscillator system which is stable even in a flowing liquid and which, in this configuration, is sensitive enough to allow bio-molecular interactions on the surface of the crystal electrode to be monitored in real time.

In another preferred embodiment, a system to monitor patient samples in real time comprises an AT cut quartz crystal oscillator attached to an RF lever oscillator drive circuit sandwiched between two O-rings within a liquid flow cell and encapsulated within an enclosure to reduce signal noise by minimizing temperature fluctuation; an electronically controlled six-port flow valve; a frequency counter; an analog to digital converter; software to analyze data. Preferably, the quartz crystal is about 100 nm thick and has a diameter of about 0.5 mm and the RF lever oscillator drive circuit has a drive frequency of at least about 10 MHz. The quartz crystal can be about 100 nm thick, 0.5 mm diameter and the thickness ranges from about 1 nm to about 1000 nm, a diameter from 0.1 to 25 mm.

The crystals can be AT, BT and AZ cut crystals for use in the system and methods of the invention.

In other preferred embodiments, the quartz crystal oscillator is substituted with fluorescence detection cell; chemoresistors; chemocapcitors; gravimetric sensor; optical refractance sensor; calorimetric sensor; amperometric sensor; or an optical absorbance.

In a preferred embodiment, the gold surface is substituted by other metals (chromium, copper, molybdenum, nickel, palladium, silicon, zinc), by carbon, by a polymeric film, by an organic film, by quartz and glass compositions.

In another preferred embodiment, the RF lever oscillator drive circuit is substituted using a standard RF clock oscillator; oscillator with automatic gain control; an oscillator circuit that measures either or both of resonant frequency change and resonant amplitude dampening or resistance; a phase-lock oscillator; or an electrochemical oscillator detection circuit.

In another preferred embodiment, the two rubber O-rings are substituted with any water tight compression fitting; or any water tight annealed fitting.

In another preferred embodiment, the styrofoam is substituted with any heat isolative material (e.g., foam, vacuum chamber, fiberglass, paper products, etc); any actively or passive controlled temperature chamber (e.g., resistive heater, compressor, peltier device).

In another preferred embodiment, the frequency counter is substituted with electronic devices for measuring minute RF cycle changes; any optical devices for measuring minute RF cycle changes; any electronic or optical devices for measuring resistive, amplitude, gravimetric, refractive, calorimetric changes associated with a physical property shift after binding of an antibody with its antigen; any electronic or optical devices for measuring resistive, amplitude, gravimetric, refractive, fluorescence, absorbance, calorimetric changes associated with a physical property shift after binding of an aptomer with its corresponding nucleic acid or amino acid partner.

In another preferred embodiment the custom software described in the Examples which follow is substituted with any custom or commercially available software for use in measuring and recording changes in frequency oscillation, resistive, amperometric, graimetric, refractive, fluorescence, calorimetric changes.

In another preferred embodiment, a system for detecting and diagnosing neural injury in a patient, said system comprises a biosensor; a flow cell for delivery of a sample from said patient to said biosensor; a capture probe adsorbed to surface of said biosensor; and, a computational system communicably connected to said biosensor, said computational system correlating the detection of one or more protein biomarkers in said sample with a diagnosis of neural injury and/or neuronal disorders, wherein the correlation takes into account the detection of one or more protein biomarkers in each diagnosis, as compared to normal subjects. Preferably, the biosensor is a quartz crystal microbalance which comprises a surface adsorbed capture molecule and the surface adsorbed capture molecule is an antibody.

In another preferred embodiment, the antibody specifically binds neural biomarkers as identified in Table 1, peptides, fragments, variants or derivatives thereof.

In another preferred embodiment, the biosensor is subjected to an equilibration step by a flow through of renewing buffer and the renewing buffer removes unbound capture molecule. Preferably, the system further comprises a reference biosensor.

In another preferred embodiment, the system further comprises a patient sample flow regulator, a patient sample flows from a patient into a flow cell and the flow cell regulates the flow rate of sample and buffers.

In another preferred embodiment, binding of the capture molecule, adsorbed to the surface of the quartz crystal microbalance, to a ligand produces a detectable resonance, wherein, the resonance is indicative of specific binding. Preferably, the resonance is between about 1 Hz up to 40 Hz. The system allows for one or more biomarkers to be detected and capture molecules of different specificities are adsorbed to addressable locations on the surface of biosensor.

In another preferred embodiment, a method of detection and identification of protein biomarkers in a patient comprises providing a patient sample; capturing of biomarkers on a substrate surface by a surface substrate bound capturing molecule; applying an oscillating electric field across the substrate surface; measuring at least one resonant frequency of the substrate surface; measuring the admittance magnitude at the resonant frequencies simultaneously, and correlating the resonant frequency and the admittance magnitude to obtain a surface mass density; thereby, detecting and identifying one or more biomarkers. Preferably, the computational system correlates surface mass density with the amount of biomarker bound by the capture molecule. The substrate surface is subjected to an equilibration step which removes any unbound molecules and the substrate surface is in contact with a buffer which optimizes binding reactions between the biomarker and capture probe. The substrate surface comprising the bound biomarker capture molecule is washed with disassociation buffer to remove biomarkers bound by the capture molecule. Preferably, the disassociation buffer disassociates the biomarker from the capture molecule without removing the capture molecule from the surface of the surface substrate and the substrate surface is washed with washing buffer to allow binding of biomarkers from a successive sample from a patient. Preferably, the substrate surface is reused in successive measurements of biomarkers in a sample.

In another preferred embodiment, a method of real-time measurement of biomarkers in a patient sample comprises the steps of equilibration; antigen association; antigen disassociation and regeneration. The equilibration step is performed after a capture molecule is adsorbed to a biosensor surface and the sample from a patient flows at a rate of about 1 ml per 10 to 30 minutes over the surface of the biosensor allowing for binding of ligand and antibody.

In a preferred embodiment, the biosensor detects at least one biomarker or a plurality of biomarkers. The data generated from the detection is acquired on a portable computer and the data provides a real-time monitoring of a patient suffering from neural injury. Preferably, the data is generated between about 0.5 minutes to about 5 minutes of the patient sample flow-through.

In another preferred embodiment, a successive patient sample comprises biomarkers that bind to capture molecules after the biosensor is washed with disassociation buffer and releasing previously bound biomarkers. Preferably, the binding of different biomarkers is diagnostic of type of neural injury, the location of the injury, and the degree of severity of the injury.

Other aspects of the invention are described infra.

### BRIEF DESCRIPTION OF THE DRAWINGS

The invention is pointed out with particularity in the appended claims. The above and further advantages of this invention may be better understood by referring to the following description taken in conjunction with the accompanying drawings, in which:

FIG. 1 shows a schematic representation of the biosensor equilibration step and the oscillating wave frequency.

FIG. 2 shows a schematic representation of the antigen association step and oscillating wave frequency.

FIG. 3 shows a schematic representation of the washing step to remove unbound ligand and the oscillating wave frequency.

FIG. 4 shows a schematic representation of the biosensor regeneration step wherein bound biomarkers are removed with disassociation buffer and the oscillating wave frequency.

FIG. 5 is a schematic general illustration of the system for real-time monitoring of biomarkers from patients suffering from neural injury. FIG. 5 depicts the equilibration step.

FIG. 6 is a schematic general illustration of the system for real-time monitoring of biomarkers from patients suffering from neural injury. FIG. 6 depicts the antigen association step.

FIG. 7 is a schematic general illustration of the system for real-time monitoring of biomarkers from patients suffering from neural injury. FIG. 7 depicts the washing/detection step.

FIG. 8 is a schematic general illustration of the system for real-time monitoring of biomarkers from patients suffering from neural injury. FIG. 8 depicts the regeneration step.

FIGS. 9A-9C are schematic illustrations showing alternative methods for the biomarker detection step. FIG. 9A is an illustration showing a competing detecting agent such as a competing antibody, against the immobilized detecting antibody. Selection is based on agents that directly or indirectly compete for binding with biomarker binding sites, that is, it will only bind free immobilized detecting antibody, but not biomarker-bound antibody. FIG. 9B is an illustration showing converting competing antibody binding to anti A/B to electrical signal by introducing surface charge to the biochip (negative) while positively charging the competing antibody (CA)⁺. FIG. 9C is an illustration showing multiple biomarker detection by various fluorophores. Different fluorophores are covalently linked to the different competing antibodies. Once bound, and after washing to remove unbound competing antibodies, lasers are used to excite the fluorophore at different wavelengths and fluorescein emission can then be detected at various wavelengths and converted to electric signal via photo multiplier tube.

FIG. 10 is an image showing a prototype continuous monitoring device based on QCM-liquid flow cell technology

FIG. 11 is an image showing a flow cell and QCM sensor used in prototype device. A 10 MHz crystal is used with a 5 mm diameter gold sensing region deposited to 100 nm thickness. The crystal is placed within a 75 μL dynamic liquid cell for continuous or stop flow monitoring.

FIG. 12 is a graph showing data collected on the prototype device. Shown is the adsorption of protein A (1 mg/mL in PBS) onto the gold surface. The initial phase shows the steady state signal for pure PBS (equilibration), then the adsorption phase where the protein A binds, then the wash phase where non-adsorbed material is removed to perform the measurement.

FIG. 13 is an image showing atomic force microscopy (AFM) data on antibody immobilization onto gold via Protein A linkage. The gold surface was pre-treated with piranha solution for 5 minutes and dried with nitrogen gas; the first AFM was captured of the clean surface (at right). 100 μL of protein A (1 mg/mL in Tris-HCl pH 7.4) was applied to the surface for 1 hour, and washed 3 times with PBS; the second AFM was captured of the protein A coating. The protein A coated chip was incubated with 100 μL of 0.25 mg/mL anti-streptavidin-ALP and immobilized by the protein A and washed 3 times in PBS. The final AFM image was captured showing the significant increase in density of the antibody—protein A is known to bind up to four antibodies per molecule. AFM images were captured on a Digital Instruments Dimension 3100 operated in contact mode over a 10×10 μm region of the gold surface.

FIG. 14 is a graph showing antigen incubation time. The absorbance of the final solution was recorded at 405 nm and plotted for each antigen incubation time in FIG. 14.

FIG. 15 is a graph showing a linear response for antigen binding. 100 μL of six streptavidin-ALP (antigen) solutions at concentrations of 0, 0.00125, 0.0025, 0.005, 0.010, 0.025 mg/mL in Tris-hydrochloride buffer (pH 7.4) were placed atop immobilized antibody treated gold surfaces for 10 minutes. Following antigen binding, the surface was washed three times with PBS. The ALP conjugate was again used to hydrolyze pNPP as described above. The solution absorbance was plotted against antigen concentration.

FIG. 16 is a graph showing short term regeneration performance of the immobilized antibody surface conducted at room temperature.

FIG. 17 is a graph showing short term regeneration performance of the immobilized antibody surface conducted at 4° C.

FIG. 18 is a graph showing long term regeneration whereby, re-incubation with streptavidin-ALP and pNPP was performed in increments of days at room temperature.

FIG. 19 is a graph showing long term regeneration whereby, re-incubation with streptavidin-ALP and pNPP was performed in increments of days at 4° C.

### DETAILED DESCRIPTION

A real-time system monitors neural injury and/or neural disorders. In particular, a biomonitor comprising a capture molecule, adsorbed to the surface of a quartz crystal microbalance, specifically binds neural biomarkers that are diagnostic of the type of neural injury, the degree and severity of neural injury, and the in vivo location of neural injury. Successive samples from a patient provide real-time data for the bed-side monitoring of patients. Further applications of the biomonitor, include biological and chemical agent detection.

The system has competitive advantages in that it provides data in real-time. It is of great importance to health care providers caring for acutely ill patients, for authorities against bioterrorism, for monitoring of fluid quality (e.g. drinking water quality, monitoring for algae blooms in coastal sea water or freshwater reserves and/or other industrial or scientific fluids that are susceptible to pathogen contamination).

Prior to setting forth the invention, the following definitions are provided. Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

### DEFINITIONS

“Marker” or “biomarker” are used interchangeably herein, and in the context of the present invention refers to a polypeptide (of a particular apparent molecular weight) which is differentially present in a sample taken from patients having neural injury and/or neuronal disorders as compared to a comparable sample taken from control subjects (e.g., a person with a negative diagnosis, normal or healthy subject).

A “plurality” refers preferably to a group of at least about 2, more preferably, at least about 5, more preferably, at least about 10, more preferably to a group of at least 100, more preferably to a group of at least 1,000 members. The maximum number of members is unlimited, but is at least 100,000 members.

“Complementary” in the context of the present invention refers to detection of at least two biomarkers, which when detected together provides increased sensitivity and specificity as compared to detection of one biomarker alone.

The phrase “differentially present” refers to differences in the quantity and/or the frequency of a marker present in a sample taken from patients having for example, neural injury as compared to a control subject. For example, a marker can be a polypeptide which is present at an elevated level or at a decreased level in samples of patients with neural injury compared to samples of control subjects. Alternatively, a marker can be a polypeptide which is detected at a higher frequency or at a lower frequency in samples of patients compared to samples of control subjects. A marker can be differentially present in terms of quantity, frequency or both.

A polypeptide is differentially present between the two samples if the amount of the polypeptide in one sample is statistically significantly different from the amount of the polypeptide in the other sample. For example, a polypeptide is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

Alternatively or additionally, a polypeptide is differentially present between the two sets of samples if the frequency of detecting the polypeptide in samples of patients' suffering from neural injury and/or neuronal disorders, is statistically significantly higher or lower than in the control samples. For example, a polypeptide is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

An advantage of assays of the invention is that they are performed in “real time”. As used herein in reference to monitoring, measurements or observations in assays of the invention, the term “real time” refers to that which is performed contemporaneously with the monitored, measured or observed events and which yields a result of the monitoring, measurement or observation to one who performs it simultaneously, or effectively so, with the occurrence of a monitored, measured or observed event. Thus, a “real time” assay or measurement contains not only the measured and quantitated result, but expresses this in real time, that is, in hours, minutes, seconds, milliseconds, nanoseconds, picoseconds, etc.

“Diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A “test amount” of a marker refers to an amount of a marker present in a sample being tested. A test amount can be either in absolute amount (e.g., μg/ml) or a relative amount (e.g., relative intensity of signals).

A “diagnostic amount” of a marker refers to an amount of a marker in a subject's sample that is consistent with a diagnosis of neural injury and/or neuronal disorder. A diagnostic amount can be either in absolute amount (e.g., μg/ml) or a relative amount (e.g., relative intensity of signals).

A “control amount” of a marker can be any amount or a range of amount which is to be compared against a test amount of a marker. For example, a control amount of a marker can be the amount of a marker in a person without neural injury and/or neuronal disorder. A control amount can be either in absolute amount (e.g., μg/ml) or a relative amount (e.g., relative intensity of signals).

“Substrate” or “probe substrate” refers to a solid phase onto which an adsorbent can be provided (e.g., by attachment, deposition, etc.).

“Adsorbent” refers to any material capable of adsorbing a marker. The term “adsorbent” is used herein to refer both to a single material (“monoplex adsorbent”) (e.g., a compound or functional group) to which the marker is exposed, and to a plurality of different materials (“multiplex adsorbent”) to which the marker is exposed. The adsorbent materials in a multiplex adsorbent are referred to as “adsorbent species.” For example, an addressable location on a probe substrate can comprise a multiplex adsorbent characterized by many different adsorbent species (e.g., anion exchange materials, metal chelators, or antibodies), having different binding characteristics. Substrate material itself can also contribute to adsorbing a marker and may be considered part of an “adsorbent.”

“Adsorption” or “retention” refers to the detectable binding between an absorbent and a marker either before or after washing with an eluant (association buffer, dissociation buffer) or a washing solution.

“Eluant” or “buffer” refers to an agent that can be used to mediate adsorption of a marker to an adsorbent. Eluants and washing solutions are also referred to as “association” or “dissociation” buffers. For example, dissociation buffers can be used to dissociate previously bound markers from the adsorbent molecules or surface (e.g. marker bound by antibody); association buffers can be used to allow for association of markers to adsorbent molecules (e.g. marker bound to antibody); washing buffer can be used to wash and remove unbound materials from the probe substrate surface.

“Resolve,” “resolution,” or “resolution of marker” refers to the detection of at least one marker in a sample. Resolution includes the detection of a plurality of markers in a sample by separation and subsequent differential detection. Resolution does not require the complete separation of one or more markers from all other biomolecules in a mixture. Rather, any separation that allows the distinction between at least one marker and other biomolecules suffices.

“Detect” refers to identifying the presence, absence or amount of the object to be detected.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

“Detectable moiety” or a “label” refers to a composition detectable by spectroscopic, photochernical, biochemical, immunochemical, or chemical means. For example, useful labels include ³²P, ³⁵S, fluorescent dyes, electron-dense reagents, enzymes (e.g., as commonly used in an ELISA), biotin-streptavidin, dioxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used tc) quantify the amount of bound detectable moiety in a sample. Quantitation of the signal is achieved by, e.g., scintillation counting, densitometry, or flow cytometry.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes anti-body fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

“Immunoassay” is an assay that uses an antibody to specifically bind an antigen (e.g., a marker). The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to marker NF-200 from specific species such as rat, mouse, or human can be selected to obtain only those polyclonal antibodies that are specifically immunoreactive with marker NF-200 and not with other proteins, except for polymorphic variants and alleles of marker NF-200. This selection may be achieved by subtracting out antibodies that cross-react with marker NF-200 molecules from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

The terms “binding component”, “molecule of interest”, “agent of interest”, “ligand” or “receptor” may be any of a large number of different molecules, biological cells or aggregates, and the terms are used interchangeably. Each binding component is immobilized at a cell, sector, site or element of the biosensor surface and binds to an analyte being detected. Therefore, the location of an element or cell containing a particular binding component determines what analyte will be bound. Proteins, polypeptides, peptides, nucleic acids (nucleotides, oligonucleotides and polynucleotides), antibodies, ligands, saccharides, polysaccharides, microorganisms such as bacteria, fungi and viruses, receptors, antibiotics, test compounds (particularly those produced by combinatorial chemistry), plant and animal cells, organelles or fractions of each and other biological entities may each be a binding component if immobilized on the chip. Each, in turn, also may be considered as analytes if same bind to a binding component on a biosensor.

The term “bind” includes any physical attachment or close association, which may be permanent or temporary. Generally, an interaction of hydrogen bonding, hydrophobic. forces, van der Waals forces, covalent and ionic bonding etc., facilitates physical attachment between the molecule of interest and the analyte being measuring. The “binding” interaction may be brief as in the situation where binding causes a chemical reaction to occur. That is typical when the binding component is an enzyme and the analyte is a substrate for the enzyme. Reactions resulting from contact between the binding agent and the analyte are also within the definition of binding for the purposes of the present invention.

“Sample” is used herein in its broadest sense. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

“Substantially purified” refers to nucleic acid molecules or proteins that are removed from their natural environment and are isolated or separated, and are at least about 60% free, preferably about 75% free, and most preferably about 90% free, from other components with which they are naturally associated.

“Substrate” refers to any rigid or semi-rigid support to which nucleic acid molecules or proteins are bound and includes quartz crystals, membranes, filters, chips, slides, wafers, fibers, magnetic or nonmagnetic beads, gels, capillaries or other tubing, plates, polymers, and microparticles with a variety of surface forms including wells, trenches, pins, channels and pores.

The term “coating” means a layer that is either naturally or synthetically formed on or applied to the surface of the substrate. For instance, exposure of a substrate, such as silicon, to air results in oxidation of the exposed surface. In the case of a substrate made of silicon, a silicon oxide coating is formed on the surface upon exposure to air. In other instances, the coating is not derived from the substrate and may be placed upon the surface via mechanical, physical, electrical, or chemical means. An example of this type of coating would be a metal coating that is applied to the biosensor surface such as gold, chromium, copper, molybdenum, nickel, palladium, silicon, zinc, carbon, a polymeric film, by an organic film, quartz and glass compositions. The surface thickness can range from about 1 to 1000 nm. Although a coating may be of any thickness, typically the coating has a thickness smaller than that of the substrate.

An “interlayer” is an additional coating or layer that is positioned between the first coating and the substrate. Multiple interlayers may optionally be used together. The primary purpose of a typical interlayer is to aid adhesion between the first coating and the substrate. One such example is the use of a titanium or chromium interlayer to help adhere a gold coating to a silicon or glass surface. However, other possible functions of an interlayer are also anticipated. For instance, some interlayers may perform a role in the detection system (such as a semiconductor or metal layer between a nonconductive substrate and a nonconductive coating).

An “organic thinfilm” is a thin layer of organic molecules which has been applied to a substrate or to a coating on a substrate if present. Typically, an organic thinfilm is less than about 20 nm thick. Optionally, an organic thinfilm may be less than about 10 nm thick. An organic thinfilm may be disordered or ordered. For instance, an organic thinfilm can be amorphous (such as a chemisorbed or spin-coated polymer) or highly organized (such as a Langmuir-Blodgett film or self-assembled monolayer). An organic thinfilm may be heterogeneous or homogeneous. Organic thinfilm which are monolayers are preferred. A lipid bilayer or monolayer is a preferred organic thinfilm. Optionally, the organic thinfilm may comprise a combination of more than one form of organic thinfilm. For instance, an organic thinfilm may comprise a lipid bilayer on top of a self-assembled monolayer. A hydrogel may also compose an organic thinfilm. The organic thinfilm will typically have functionalities exposed on its surface which serve to enhance the surface conditions of a substrate or the coating on a substrate in any of a number of ways. For instance, exposed functionalities of the organic thinfilm are typically useful in the binding or covalent immobilization of the protein-capture agents to the patches of the array. Alternatively, the organic thinfilm may bear functional groups (such as polyethylene glycol (PEG)) which reduce the non-specific binding of molecules to the surface. Other exposed functionalities serve to tether the tinfoil to the surface of the substrate or the coating. Particular functionalities of the organic thinfilm may also be designed to enable certain detection techniques to be used with the surface. Alternatively, the organic thinfilm may serve the purpose of preventing inactivation of a protein-capture agent or the protein to be bound by a protein-capture agent from occurring upon contact with the surface of a substrate or a coating on the surface of a substrate.

A “monolayer” is a single-molecule thick organic thinfilm. A monolayer may be disordered or ordered. A monolayer may optionally be a polymeric compound, such as a polynonionic polymer, a polyionic polymer, or a block-copolymer. For instance, the monolayer may be composed of a poly(amino acid) such as polylysine. A monolayer which is a self-assembled monolayer, however, is most preferred. One face of the self-assembled monolayer is typically composed of chemical functionalities on the termini of the organic molecules that are chemisorbed or physisorbed onto the surface of the substrate or, if present, the coating on the substrate if present. Examples of suitable functionalities of monolayers include the positively charged amino groups of poly-L-lysine for use on negatively charged surfaces and thiols for use on gold surfaces. Typically, the other face of the self-assembled monolayer is exposed and may bear any number of chemical functionalities (end groups). Preferably, the molecules of the self-assembled monolayer are highly ordered.

A “self-assembled monolayer” is a monolayer which is created by the spontaneous assembly of molecules. The self-assembled monolayer may be ordered, disordered, or exhibit short- to long-range order.

An “affinity tag” is a functional moiety capable of directly or indirectly immobilizing a protein-capture agent onto an exposed functionality of the organic thinfilm. Preferably, the affinity tag enables the site-specific immobilization and thus enhances orientation of the protein-capture agent onto the organic thinfilm. In some cases, the affinity tag may be a simple chemical functional group. Other possibilities include amino acids, poly(amino acid) tags, or full-length proteins. Still other possibilities include carbohydrates and nucleic acids. For instance, the affinity tag may be a polynucleotide which hybridizes to another polynucleotide serving as a functional group on the organic thinfilm or another polynucleotide serving as an adaptor. The affinity tag may also be a synthetic chemical moiety. If the organic thinfilm of each of the patches comprises a lipid bilayer or monolayer, then a membrane anchor is a suitable affinity tag. The affinity tag may be covalently or noncovalently attached to the protein-capture agent. For instance, if the affinity tag is covalently attached to the protein-capture agent it may be attached via chemical conjugation or as a fusion protein. The affinity tag may also be attached to the protein-capture agent via a cleavable linkage. Alternatively, the affinity tag may not be directly in contact with the protein-capture agent. The affinity tag may instead be separated from the protein-capture agent by an adaptor. The affinity tag may immobilize the protein-capture agent to the organic thinfilm either through noncovalent interactions or through a covalent linkage.

An “adaptor”, for purposes of this invention, is any entity that links an affinity tag to the protein-capture agent. The adaptor may be, but need not necessarily be, a discrete molecule that is noncovalently attached to both the affinity tag and the protein-capture agent. The adaptor can instead be covalently attached to the affinity tag or the protein-capture agent or both (via chemical conjugation or as a fusion protein, for instance). Proteins such as full-length proteins, polypeptides, or peptides are typical adaptors. Other possible adaptors include carbohydrates or nucleic acids.

The term “fusion protein” refers to a protein composed of two or more polypeptides that, although typically unjoined in their native state, are joined by their respective amino and carboxyl termini through a peptide linkage to form a single continuous polypeptide. It is understood that the two or more polypeptide components can either be directly joined or indirectly joined through a peptide linker/spacer.

As used herein, the term “injury or neural injury” is intended to include a damage which directly or indirectly affects the normal functioning of the CNS. For example, the injury can be damage to retinal ganglion cells; subarachnoid hemorrhage, epilepsy, stroke, and other forms of brain injury, a traumatic brain injury; a stroke related injury; a cerebral aneurism related injury; a spinal cord injury, including monoplegia, diplegia, paraplegia, hemiplegia and quadriplegia; a neuroproliferative disorder or neuropathic pain syndrome. Examples of CNS injuries or disease include TBI, stroke, concussion (including post-concussion syndrome), cerebral ischemia, neurodegenerative diseases of the brain such as Parkinson's disease, Dementia Pugilistica, Huntington's disease and Alzheimer's disease, Creutzfeldt-Jakob disease, brain injuries secondary to seizures which are induced by radiation, exposure to ionizing or ion plasma, nerve agents, cyanide, toxic concentrations of oxygen, neurotoxicity due to CNS malaria or treatment with anti-malaria agents, and other CNS traumas.

As used herein, the term “Traumatic Brain Injury” (TBI) is art recognized and is intended to include the condition in which, a traumatic blow to the head causes damage to the brain, often without penetrating the skull. Usually, the initial trauma can result in expanding hematoma, subarachnoid hemorrhage, cerebral edema, raised intracranial pressure (ICP), and cerebral hypoxia, which can, in turn, lead to severe secondary events due to low cerebral blood flow (CBF).

“Neural cells” as defined herein, are cells that reside in the brain, central and peripheral nerve systems, including, but not limited to, nerve cells, glial cell, oligodendrocyte, microglia cells or neural stem cells.

“Neuronal specific or neuronally enriched proteins” are defined herein, as proteins that are present in neural cells and not in non-neuronal cells, such as, for example, cardiomyocytes, myocytes, in skeletal muscles, hepatocytes, kidney cells and cells in testis.

“Neural (neuronal) defects, disorders or diseases” as used herein refers to any neurological disorder, including but not limited to neurodegenerative disorders (Parkinson's; Alzheimer's) or autoimmune disorders (multiple sclerosis) of the central nervous system; memory loss; long term and short term memory disorders; learning disorders; autism, depression, benign forgetfulness, childhood learning disorders, close head injury, and attention deficit disorder; autoimmune disorders of the brain, neuronal reaction to viral infection; brain damage; depression; psychiatric disorders such as bi-polarism, schizophrenia and the like; narcolepsy/sleep disorders (including circadian rhythm disorders, insomnia and narcolepsy); severance of nerves or nerve damage; severance of the cerebrospinal nerve cord (CNS) and any damage to brain or nerve cells; neurological deficits associated with AIDS; tics (e.g. Giles de la Tourette's syndrome); Huntington's chorea, schizophrenia, traumatic brain injury, tinnitus, neuralgia, especially trigeminal neuralgia, neuropathic pain, inappropriate neuronal activity resulting in neurodysthesias in diseases such as diabetes, MS and motor neuron disease, ataxias, muscular rigidity (spasticity) and temporomandibular joint dysfunction; Reward Deficiency Syndrome (RDS) behaviors in a subject.

The term “closed head injury,” as used herein, refers to a clinical condition after head injury or trauma which condition can be characterized by cognitive and memory impairment. Such a condition can be diagnosed as “amnestic disorder due to a general medical condition” according to DSM-IV.

As used herein, “subcellular localization” refers to defined subcellular structures within a single nerve cell. These subcellularly defined structures are matched with unique neural proteins derived from, for example, dendritic, axonal, myelin sheath, presynaptic terminal and postsynaptic locations. By monitoring the release of proteins unique to each of these regions, one can therefore monitor and define subcellular damage after brain injury. Furthermore, mature neurons are differentiated into dedicated subtype fusing a primary neural transmitter such as cholinergic (nicotinic and mucarinic), glutamatergic, gabaergic, serotonergic, dopaminergic. Each of this neuronal subtype express unique neural proteins such as those dedicated for the synthesis, metabolism and transporter and receptor of each unique neurotransmitter system.

As used herein, a “pharmaceutically acceptable” component is one that is suitable for use with humans and/or animals without undue adverse side effects (such as toxicity, irritation, and allergic response) commensurate with a reasonable benefit/risk ratio.

The terms “patient” or “individual” are used interchangeably herein, and is meant a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

As used herein, “ameliorated” or “treatment” refers to a symptom which is approaches a normalized value, e.g., is less than 50% different from a normalized value, preferably is less than about 25% different from a normalized value, more preferably, is less than 10% different from a normalized value, and still more preferably, is not significantly different from a normalized value as determined using routine statistical tests. For example, amelioration or treatment of depression includes, for example, relief from the symptoms of depression which include, but are not limited to changes in mood, feelings of intense sadness and despair, mental slowing, loss of concentration, pessimistic worry, agitation, and self-deprecation. Physical changes may also be relieved, including insomnia, anorexia and weight loss, decreased energy and libido, and the return of normal hormonal circadian rhythms. Another example, when using the terms “treating Parkinson's disease” or “ameliorating” as used herein means relief from the symptoms of Parkinson's disease which include, but are not limited to tremor, bradykinesia, rigidity, and a disturbance of posture.

By “charge coupled device,” also referred to as CCD, is meant a well-known electronic device which outputs an electrical signal proportional to the incident energy upon the CCD surface in a spatially addressable manner.

The term “CCD proximal detection” refers to the use of CCD technology for detection and imaging in which the CCD is proximal to the sample to be analyzed, thereby avoiding the need for conventional lenses.

A “biosensor” generally refers to a small, portable, analytical device based on the combination of recognition biomolecules with an appropriate transducer, and which detects chemical or biological materials selectively and with high sensitivity. In accordance with the invention, a “biosensor” detects at least one biomarker and/or a plurality of biomarkers. Other examples include, but are not limited to: detecting toxic substances from a variety of sources such as air, water or soil samples or may be used to monitor enclosed environments. They also may be formulated as catheters for monitoring drug and metabolite levels in vivo, or as probes for the analysis of toxic substances, drugs or metabolites in samples of say, blood and urine.

“Ligands” refers to molecules which are recognized by a particular receptor. “Ligands” may include, without limitation, agonists and antagonists for cell membrane receptors, toxins, venoms, oligosaccharides, proteins, bacteria and monoclonal antibodies.

By “throughput” is meant the number of analyses completed in a given unit of time.

### Real-Time Moizitorinzg of Biomarkers—Quartz Crystal Microbalance (QCM)

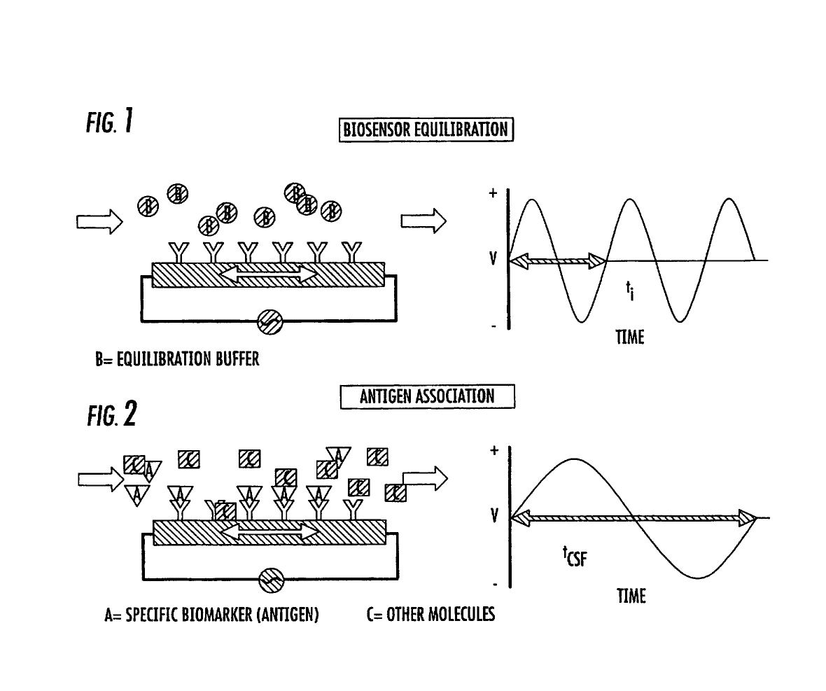

The invention provides an on-line monitoring sensor or “lab-on-a-chip” or biosensor which is, preferably, connected to a biological fluid flow directly from a patient. The patient sample is drawn, for example, from a cerebral spinal fluid (CSF) drainage system (one-way), an arterial line (blood), brain microdialysate, hemodialysate, or other biofluids (FIG. 1).

In a preferred embodiment, the biomonitor is placed at the bed side to record biomarker concentrations on a continual automated basis. Preferred biomarkers are identified by Table 1 and include, peptides, fragments, variants and derivatives thereof. The device is a diagnostic tool providing both instantaneous readings and a historical record for prompting appropriate treatment. Clinical records of TBI patients can be compared in later review to relate biomonitor data with patient outcome.

As an illustrative example, not meant to limit or construe the invention in any way, the following is provided. The QCM is treated with a capture probe which adsorbs to the surface of the QCM. The QCM is equilibrated with renewing buffer and/or a washing buffer to remove any material not bound to the QCM surface. The reference QCM is set-up to provide a baseline. Appropriate controls are also adsorbed onto the QCM to eliminate any non-specific binding. A normal peristaltic pump is used to control the flow rate. A catheter needle is inserted into a patient, for example, intravenously, spinal tap and the like. Sample flows from the patient at a regulated flow rate as determined by the medical provider, for example, flow rates from a patient can be similar to flow rates of intravenous drips. The sample from a patient enters a flow cell which also controls the volume of sample and flow rate of sample leading to the QCM. Changes in mass are detected and compared to the changes in mass with the reference QCM. Data is collected and analyzed by an appropriate algorithm. Sample that has passed through the QCM is collected and stored, if desired, for future reference.

The system is preferably, re-used for detection of other biomarkers present in a patient sample. Prior to a new a sample, the QCM crystal can be subjected to a regeneration step. The entire system is washed with washing buffer and bound markers are eluted and the system is washed again. In a preferred embodiment, the regeneration of the system is about 5 minutes, more preferably, the regeneration of the system is about 3 minutes, more preferably, the regeneration of the system is about 2 minutes, more preferably, the regeneration of the system is about thirty seconds.

Once the system has been flushed with the renewing buffer and the washing buffer, the patient sample continues to flow. The sample can be processed by the QCM at time intervals desired by the operator. For example, if the injury appears to be life-threatening, then samples are taken at shorter time intervals to maximize the information, for example, to diagnose whether there has been severe damage, the type of damage, the location of the damage and the progression of the injury. This will provide a medical practitioner to make split-second decisions as to the medical procedures that need to be made to avoid further injury and to stabilize the patients condition. If, however, the patient is stabilized the time interval between different samples monitored may be longer.

In one preferred embodiment of the invention, the apparatus for real-time monitoring of biomarkers comprises a quartz crystal microbalance (QCM). The quartz crystal microbalance is a device for detecting and measuring very small changes in mass. Preferably, the QCM comprises primary components, such as a quartz crystal and an oscillator circuit coupled to the quartz crystal to produce an output at a resonant frequency of the crystal. The output frequency, is preferably between about 1 MHz to about 30 MHz, more preferably, the output frequency is 1 MHz, more preferably, the output frequency is about 10 MHz, more preferably, the output frequency is about 20 MHz, measured to a high degree of accuracy, for example, with a frequency counter. The quartz crystal is, unlike the crystals normally used in electronic circuits, unencapsulated, so that it can interact with its environment. The deposition of small quantities of material onto the crystal changes its resonant frequency and allows the determination of the mass of material deposited. Preferably, frequency changes are of the order of about 1 Hz to about 40 Hz and changes of the order of nanograms in the mass of material deposited can be detected.

Quartz has the advantages of being chemically unreactive and insoluble in water, as well as being relatively temperature insensitive. The quartz crystal is typically a circular “AT-cut” with metal electrodes on opposing faces, but the cut and shape of the crystal can include, squares, rectangles and the like. The electrodes are preferably sputtered thin (about 200 nm) films of gold, silver, or titanium, and, optionally, with a sub-layer for improved adhesion. Lead wires attach to the electrodes and also provides mechanical support for the crystal, as well as some degree of isolation from the base of the sensor and lead out wires. The crystal is about 1 cm in diameter. The change in resonant frequency, ΔF, of, for example, an AT-cut quartz crystal of area (A) vibrating in air at fundamental frequency, F, when the mass of the crystal is changed by ΔM, is given approximately by:

ΔF=−2.3×10⁶(F²ΔM/A).

The quartz crystal microbalance is most frequently used to measure mass, but can also be used to detect changes in the viscosity and/or density of a liquid, since when vibrating in a liquid all these factors effect the vibrational frequency. Thus, the shift in frequency of a quartz crystal on immersion in a liquid, ΔF, is given by:

ΔF=−F₀^{3/2}(ε_{L}ρ_{L}/πμ_{Q}ρ_{Q})^{1/2}

Where: ΔF=Change in Frequency; F₀=Resonance Frequency; ε_{L}ρ_{L}=Liquid absolute density and viscosity; μ_{Q}ρ.=Quartz elastic modulus and density.

The quartz crystal microbalance of the invention, is used as a bio-sensor, i.e. as a device which uses as part of the sensor, or is sensitive to, material of biological origin, such as a sample from a patient with traumatic brain injury. Typically, part of one or both electrodes of the sensor are coated with material which is capable of binding with a target bio-molecule or cell. When such a receptor is exposed to the target (“ligand”) compound, the ligand is bound to the substrate causing a change in mass ΔM of the sensor, and/or viscosity/density changes in the local microenvironment and a consequent change in its vibrational frequency.

The quartz crystal can be about 100 nm thick, 0.5 mm diameter and the thickness ranges from about 1 nm to about 1000 nm, a diameter from 0.1 to 25 mm.

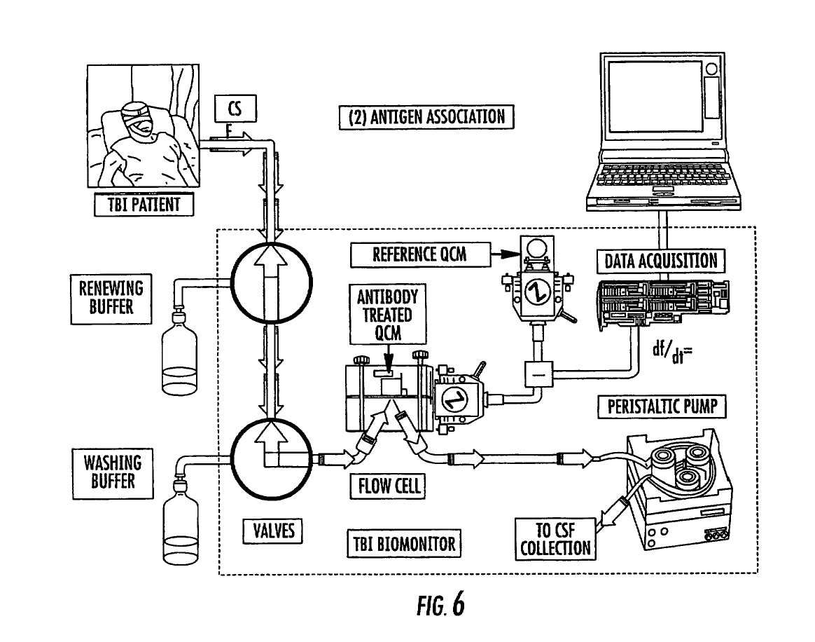

The apparatus is sensitive, among other things, to the density and viscosity of a liquid in which the sensor is immersed and can detect small mass changes even where a large tare is necessary to take account of initially deposited material. Preferably, constructed circuits are stable to ¼ Hz/day, obviating a need for a reference crystal. However, a reference crystal, as shown in FIG. 5 can be used. Also preferred, is the apparatus detect the binding of ligands of smaller molecular weight, such as for example, molecular weight less than about 600 KD, preferably, ligands of molecular weight circa 180 KD or less, such as glucose.

Quartz crystal exhibits a natural resonant frequency that is a function of the mass and structure of the crystal. The precise size, type of cut, and thickness of the quartz crystal substrate are selected to result in a particular resonant frequency. For example, an AT-cut crystal with a nominal resonant frequency of 8-30 megahertz is suitable for gas sensor applications. Suitable quartz crystal substrates may be obtained from Standard Crystal Corporation of California. Other types of suitable materials to serve as the substrate include lithium niobate (LiNbO₃), which is particularly suited for a surface acoustic wave (SAW) based-sensor; and aluminum nitride (AlN), which is particularly suited for a thin film resonator based-sensor.

The crystals can be AT, BT and AZ cut crystals for use in the system and methods of the invention.

As indicated above, the resonant frequency of the sensor is a function of the total mass of the device. Therefore, the mass of any coating provided around the crystal substrate also affects the total mass of the device, and thereby affects the resonant frequency of the sensor. The coatings provided about the crystal substrate are selected to adsorb molecules of the analyte. When analyte molecules are adsorbed by the coating, the mass of the coating is slightly increased, which in turn increases the mass of the entire sensor, and thus changes the resonant frequency of the sensor. The resonant frequency of the sensor is also a function of the visco-elastic properties of the coatings and mechanical stresses caused by temperature effects in the sensor mounting structure. However, these effects are either negligible or can be compensated for. Thus, a very sensitive sensor may be constructed by selecting a coating that has an affinity with the particular capture molecules of interest. The quantity of molecules adsorbed and deposited, and the resulting change in the operating frequency of the oscillator circuit, is a function of the concentration of the analyte being measured in the environment surrounding the sensor. This can provide the baseline measurements of a QCM with a capture molecule adsorbed, thereto. When a sample from a patient is used for diagnosis of neural injury and/or neural disorders, the changes in mass result in frequency changes in a linear fashion. Thus, a change in the mass may be measured by measuring the change in the frequency of the oscillator output. The sensor may be calibrated by exposing the sensor to known concentrations of the capture molecule and known biomarker and recording the resulting frequency of the oscillator output. The sensor may then be used to measure the absolute mass of bound biomarkers comparing the measured frequency to the aforementioned recorded values.

In other preferred embodiments, instead of a quartz crystal oscillator, a. fluorescence detection cell; chemoresistors; chemocapcitors; gravimetric sensor; optical refractance sensor; calorimetric sensor; amperometric sensor; or an optical absorbance cell can be used.

The system, disclosed herein, provides a real-time monitoring with little or no error rate as compared to immunoassays. A typical immunoassay involves detecting the binding of an antigen to an antibody, or of a receptor to a ligand. For example, one immunoassay process, known as the Bayston test (Bayston R., J. Clin. Path., 25 (1972) 718-720), relies on the antibody-antigen binding process in solution to form aggregates or clumps producing a visible precipitate. This process can be monitored using a quartz crystal microbalance, which is thought to respond to changes in the viscosity and density of the medium supporting the antibody and antigen. These changes cause a drop in the resonant frequency of the sensor, the largest drop occurring at the point of full agglutination and hence a quartz crystal microbalance simplifies detection of the titer of agglutination. Optionally, antibodies to biomarkers resulting from injury can also be detected if desired, by the methods and compositions disclosed herein, and the antibody titer is directly proportional to the amount and type of biomarker present in a patient as compared to a normal individual. However, in the preferred embodiments, antibodies specific for neural injury biomarkers are adsorbed to the QCM surface and bind to neural injury biomarkers from a patient sample.

The microbalance also has the required sensitivity and robustness for a real time monitoring system to allow continuous assay of a sample, for example, to determine the amount of a biomarker in a solution. In such a configuration, the shift in resonant frequency of the sensor is related to the concentration of antibody-biomarker binding, with a roughly linear relationship between the frequency shift and a logarithm of the bound antibody-biomarker concentration.

In another preferred embodiment, a rapid equilibration-biomarker association-washing/detection-regeneration-cycle is provided to allow rapid on-line real-time monitoring. An illustrative example is shown in FIGS. 5-8. Currently, there are no technologies that permit continuous real-time monitoring of biomarkers for patients with such conditions.

Real time monitoring device (see FIG. 5): Detection agents, for example, antibodies, whole or fragments, single chain, recombinant forms, aptamers, etc) can be immobilized on the sensor chip (such as a metal, organic, or inorganic monolayer coated quartz crystal microbalance (QCM) for binding (detection of individual biomarkers). More than one biomarker could be detected and monitored simultaneously either by multiple coatings on a single sensor or operation of multiple coated sensors in series or parallel.

In a preferred embodiment, the method includes, but is not limited to the steps of: (1) association of surface molecule with ligand; (2) washing and detection; (3) regeneration; (4) equilibration of the QCM.

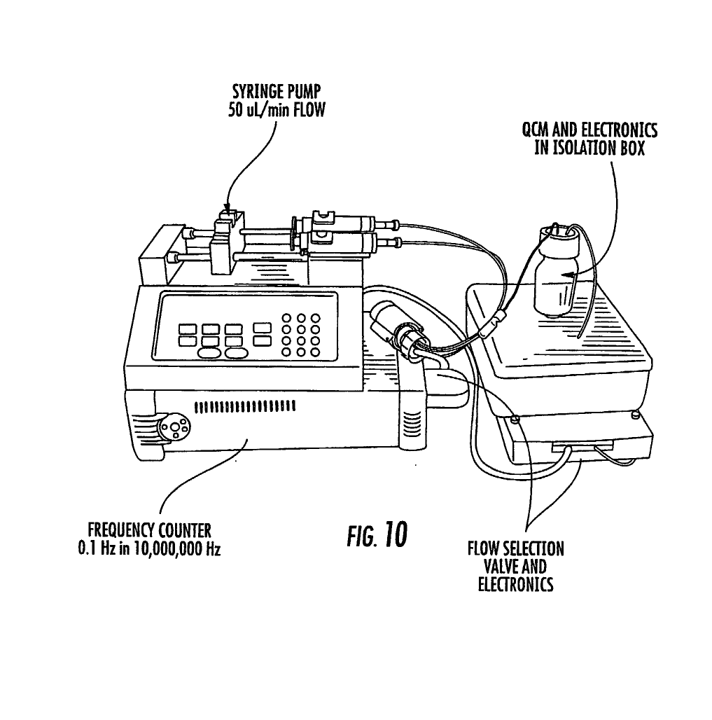

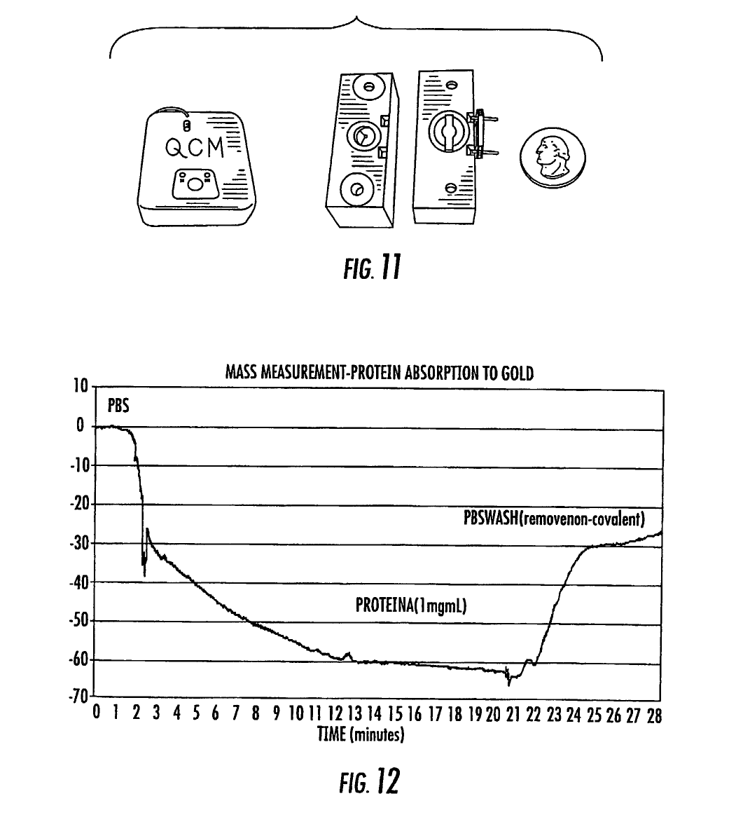

As an illustrative example, not meant to limit or construe the invention in any way, the following example of a working real time system is presented. A prototype biomonitor device was assembled as illustrated (FIG. 10). The device entails use of commercially available (International Crystal Manufacturing Company, Inc.) AT cut quartz crystal oscillator with a 100 nm thick, 0.5 mm diameter gold surface (FIG. 11). The quartz crystal is made to operate by attachment to an RF lever oscillator drive circuit that has a nominal drive frequency of 10 MHz and is commercially available from the same company. The quartz crystal is sandwiched between two rubber o-rings within a 75 μL liquid flow cell (FIG. 11) that permits contact of a fluid stream with the gold surface. The quartz crystal, flow cell, and oscillator circuitry are encapsulated within a styrofoam enclosure to reduce signal noise by minimizing temperature fluctuation. An electronically controlled six-port Rheodyne flow valve is used to deliver a user selected buffer or sample to the flow cell. A Harvard Apparatus syringe pump was used to provide a flowrate of 50 μL/min. The quartz crystal resonating frequency and amplitude are monitored by use of an Agilent frequency counter and a National Instruments analog-to-digital converter, respectively. The change from initial oscillation frequency and amplitude are recorded and displayed using custom software to derive change in surface adsorption.

A 1 mg/mL solution of protein A in phosphate buffered saline (PBS) was used to test the response of the prototype biomonitor under flow conditions. The gold surface of the quartz crystal was pre-treated with piranha solution for five minutes and dried with nitrogen gas. After the quartz crystal was inserted into the flow cell, the system was equilibrated with PBS for 5 minutes at 50 μL/min. Data acquisition is begun while continuing the PBS flow, at which point the initial oscillation frequency and amplitude are set. After one minute, the flow selection valve is switched to pass the protein A solution through the flow cell at 50 μL/min (FIG. 11). A change in frequency reflected the adsorption of protein A onto the gold surface, forming a monolayer within approximately one minute. The adsorption kinetics then changed to reflect the formation of secondary layers atop of the initial monolayer. After 20 minutes, the fluid stream was switched back to PBS in order to wash the surface of weakly bound protein A and contaminants. An increased frequency reflected removal of the secondary molecular layers, leaving behind the gold-adhered monolayer. The final frequency change of −30 Hz reflects the adsorption of the protein A monolayer.

Atomic force microscopy (AFM) images were collected to characterize the immobilization of an antibody onto a 100 nm thick gold surface via Protein A linkage (FIG. 13). The gold surface of a quartz crystal was pre-treated with piranha solution for five minutes and dried with nitrogen gas. An AFM image of the clean surface was captured (FIG. 13 at right). Next, 100 μL of a protein A solution (1 mg/mL in Tris-HCl pH 7.4) was applied to the surface for one hour, and washed 3 times with PBS; a second AFM image of the protein A coated gold was captured. Finally, 100 μL of a 0.25 mg/mL anti-streptavidin-ALP solution was added to the protein A coated gold surface for one hour and washed three times in PBS. The final AFM image was captured showing the significant increase in density of the antibody—protein A is known to bind up to four antibodies per molecule. AFM images were captured on a Digital Instruments Dimension 3100 operated in contact mode over a 10×10 μm region of the gold surface.

Antibody immobilization: A 1 mg/mL protein A in PBS solution (100 μL) was incubated atop the 100 nm thick gold surface for one hour. Following the incubation, the surface was washed 3 times (10 minutes each) with PBS. Atop the protein A coated gold was placed 100 μL of a 0.25 mg/mL anti-streptavidin solution in Tris-hydrochloride buffer (pH 7.4). After one hour of incubation, the surface was washed three times with PBS (10 minutes each). The stability of the antibody-protein A complex was strengthened by crosslinking the two using a 30 mM solution of 1,5 dimethylpimelimidate dihydrochloride in a triethanol amine buffer (pH 8.5). Non-crosslinked components were washed from the surface by washing with a glycine hydrochloride buffer (pH 2.3) for 3 minutes. Afterward the surface was rinsed 3 times for ten minutes with PBS.

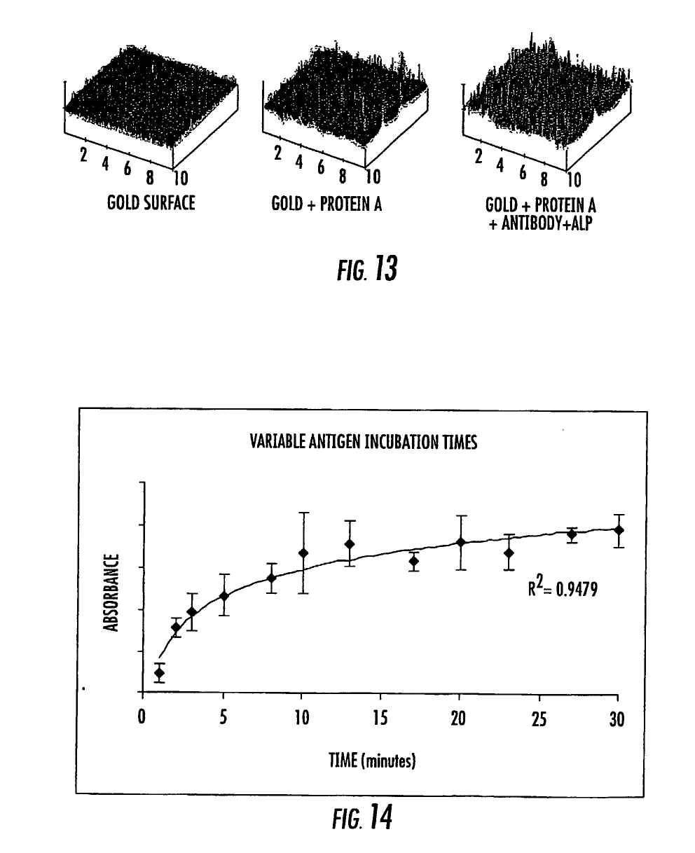

Antigen incubation time: In an experiment conducted in triplicate, immobilized antibody treated gold surfaces were incubated with 0025 mg/mL solutions of streptavidin-alkalinephosphatase (antigen) in Tris-hydrochloride buffer (pH 7.4) for 1, 2, 3, 5, 7, 10, 13, 15, 17, 20, 23, 27, and 30 minutes. The relative quantity of antigen binding was recorded by reacting the surface with a 125 μL solution of 1 mg/mL para-nitrophenylphosphate (pNPP) (pH 10). The alkalinephosphatase (ALP) hydrolyzed the pNPP within 12.5 minutes after which the solution was aspirated and mixed with 25 μL of 3M NaOH to terminate the reaction. The absorbance of the final solution was recorded at 405 nm and plotted for each antigen incubation time in FIG. 14. The time: antigen binding kinetics illustrated in FIG. 14 suggest that an endpoint of 90% binding is reached within approximately 10 minutes. Though the exponential nature of the kinetics suggests that this is a second order reaction, and thus is susceptible to the concentration of both the antigen and the antibody.

Calibration curve: In a set of three replicate experiments, 100 μL of six streptavidin-ALP (antigen) solutions at concentrations of 0, 0.00125, 0.0025, 0.005, 0.010, 0.025 mg/mL in Tris-hydrochloride buffer (pH 7.4) were placed atop immobilized antibody treated gold surfaces for 10 minutes (the previous optimized time). Following antigen binding, the surface was washed three times with PBS. The ALP conjugate was again used to hydrolyze pNPP as described above. The solution absorbance was plotted against antigen concentration in FIG. 15, which demonstrated a linear response of the immobilized antigen surface within the tested concentration range.

In other preferred embodiments, the quartz crystal oscillator is substituted with a fluorescence detection cell; chemoresistors; chemocapcitors; gravimetric sensor; optical refractance sensor; calorimetric sensor; amperometric sensor; or an optical absorbance cell

In a preferred embodiment, the gold surface is substituted by other metals (chromium, copper, molybdenum, nickel, palladium, silicon, zinc), by carbon, by a polymeric film, by an organic film, by quartz and glass compositions.

In another preferred embodiment, the surface is substituted with polystyrene surfaces, polystyrene latex particles. J. Reticuloendothelial Society 3: 29040, 1966.); Perfluorocarbon (such as fluorocarbon polymers known as Teflon™), including polytetrafluoroethylene (PTFE), polyvinylfluoride, poluvinylidene difluoride and perfluorodecalin, surfaces bind proteins or other biological molecules (U.S. Pat. No. 5,270,193). Such surfaces can be made to include fluorinated surfactants, which may render the surface hydrophilic, or positively or negatively charged. Glass, including controlled pore glass, may be modified to allow covalent attachment of antibodies, antigens, polysaccharides, polynucleotides, nucleic acids and the like. Plastic surfaces may be modified non-specifically using corona plasma discharge or electron beam radiation and then may be coated with a variety of coatings or adhesives to which macromolecules may be attached. More specific covalent attachment of biological molecules may be achieved by a variety of modifications, which attach reactive groups to polystyrene, or acrylic surfaces, which groups, with or without extending linkers, then will couple under mild conditions to the biopolymers.

In another preferred embodiment, the RF lever oscillator drive circuit is substituted using a standard RF clock oscillator; oscillator with automatic gain control; an oscillator circuit that measures either or both of resonant frequency change and resonant amplitude dampening or resistance; a phase-lock oscillator; or an electrochemical oscillator detection circuit.

In another preferred embodiment, the two rubber O-rings is substituted with any water tight compression fitting; or any water tight annealed fitting.

In another preferred embodiment, the styrofoam is substituted with any heat isolative material (e.g., foam, vacuum chamber, fiberglass, paper products, etc); any actively or passive controlled temperature chamber (e.g., resistive heater, compressor, peltier device).

In another preferred embodiment, the frequency counter is substituted with electronic devices for measuring minute RF cycle changes; any optical devices for measuring minute RF cycle changes; any electronic or optical devices for measuring resistive, amplitude, gravimetric, refractive, calorimetric changes associated with a physical property shift after binding of an antibody with its antigen; any electronic or optical devices for measuring resistive, amplitude, gravimetric, refractive, fluorescence, absorbance, calorimetric changes associated with a physical property shift after binding of an aptomer with its corresponding nucleic acid or amino acid partner.

In another preferred embodiment the custom software described in the Examples which follow is substituted with any custom or commercially available software for use in measuring and recording changes in frequency oscillation, resistive, amperometric, graimetric, refractive, fluorescence, calorimetric changes.

Nonspecific interactions: Two experiments (n=3) were conducted to assess nonspecific binding with the treated gold surface. In the first, the gold surface was coated with protein A and in the second protein A coated gold was treated with anti-actin antibody as a non-specific compliment to the streptavidin-ALP antigen. In the first experiment, the gold surfaces were treated with protein A as described earlier, incubated with streptavidin-ALP (0.025 mg/ml, 100 μl) for 10 minutes, and reacted with pNPP for 12.5 minutes with the absorbance read at 405 nm. The plates were then stored at 4° C. overnight for 4 days with the antigen binding procedure repeated each day. The average absorbance reading for each day remained constant at 0.08 absorbance units. In the second experiment (n=7), the gold surface was treated with protein A as described earlier then incubate d with 100 μL solution of anti-actin (0.75 mg/mL in Tris-hydrochloride buffer). After the incubation with the antibody, the plates were incubated with crosslinker and washed as described earlier. After incubation with streptavidin-ALP for 10 minutes and reaction with pNPP for 12.5 minutes, and the absorbance was read. The reading was 0.09 absorbance units. Together these experiments indicated a non-specific absorbance reading of <0.1 absorbance units using the pNPP detection procedure, which remained constant over 4 days of treatment.

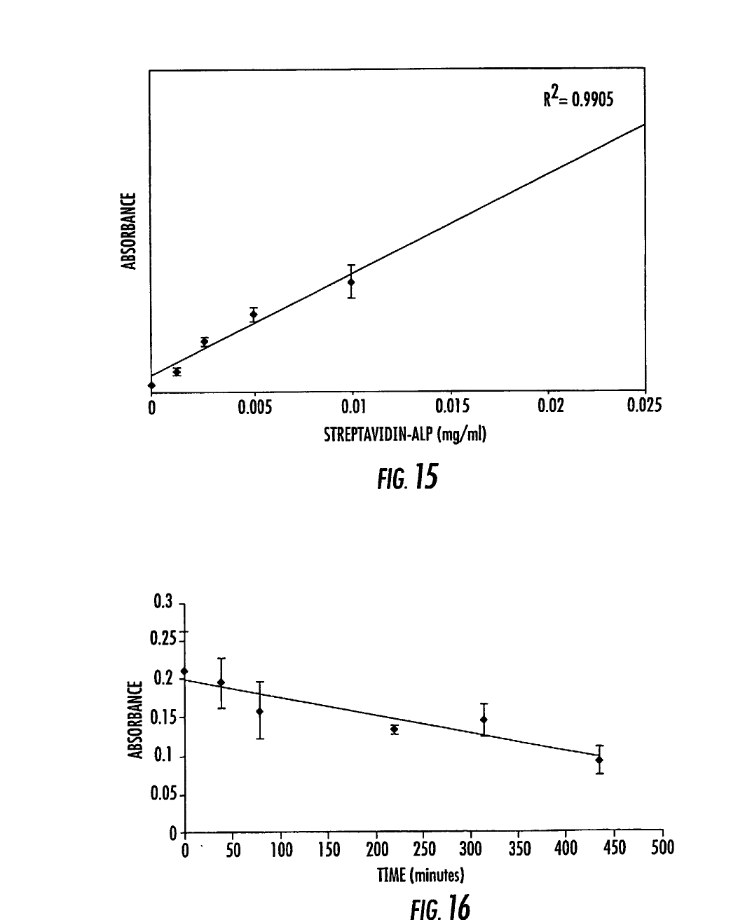

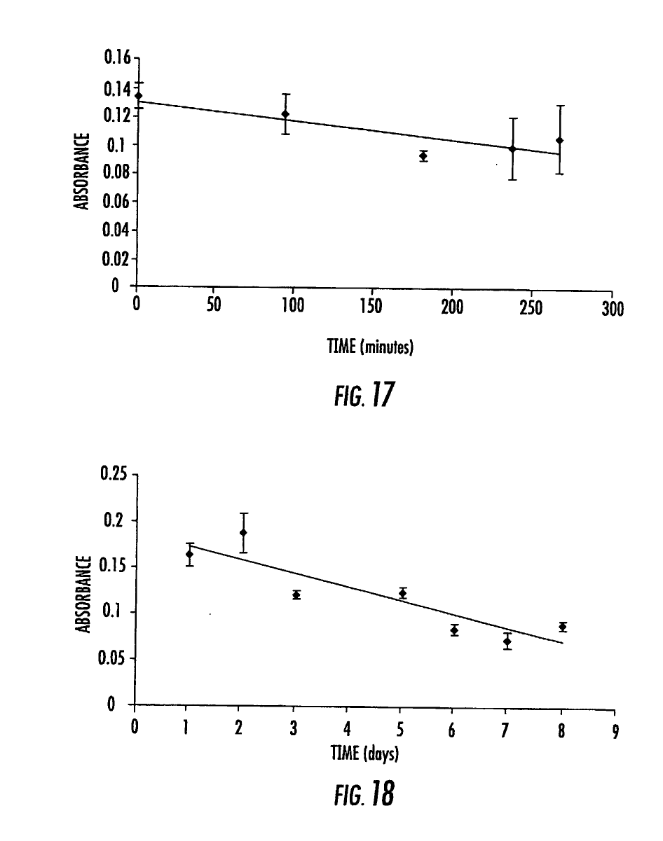

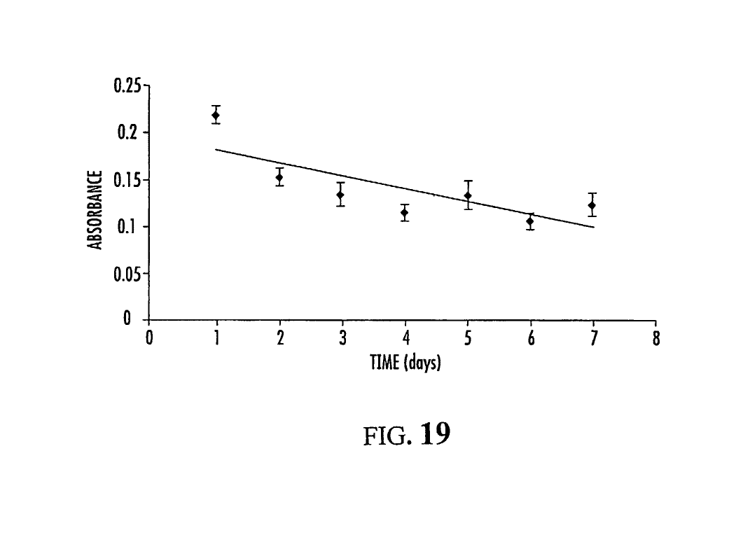

Plate regeneration: Four separate experiments were conducted to evaluate the short term and long term regeneration performance of the immobilized antibody surface at room temperature and 4° C. The gold surface was treated with protein A, anti-streptavidin, and crosslinker as described earlier. Gold surfaces (n=3) were then incubated with streptavidin-ALP at room temperature and 4° C., and reacted with pNPP for absorbance measurement. Afterwards, chips were treated with glycine-hydrochloride buffer (pH 2.3) for 3 minutes and washed with PBS. In the short term experiments, re-incubated with streptavidin-ALP and pNPP was performed in increments of minutes and hours (FIGS. 16 and 17). In the long term experiments, re-incubation with streptavidin-ALP and pNPP was performed in increments of days (FIGS. 18 and 19). The results show that regeneration is possible for three to four cycles before the absorbance approaches that of non-specific interactions. Each cycle shows a drop in absorbance for the same antigen concentration, whether at long term, short term, room temperature or 4° C. conditions. These data suggest that the immobilized antibody—protein A complex is not sufficiently stabile for regeneration by glycine-hydrochloride treatment.

New immobilization techniques: Based on the non-covalent nature of protein A binding with the gold surface, it is expected that this is the weakest point in the antibody immobilization process that prevents effective repeated measurements. The gold or other type of metal surface can be treated with biotin and coated with streptavidin as suggested by Peluso et al. (Analytical Biochemistry 2003, v312:113-124). The biomarker antibody can then be biotinylated on its F_{c} portion and bound to the streptavidin coated gold surface without the use of protein A.

As also described by Peluso et al. in the same manuscript, an antibody can be disassembled to remove the F_{c} portion from the F_{ab} portion. Then the disulfide bond between the two halves of the F_{ab} portion can be reduced to leave free thiol groups. This procedure can be performed with the biomarker antibody. Different from that presented by Peluso et al. is the concept that this newly formed thiol groups at the end of the Fₐ and F_{b} antibody portions, which is opposed to the binding end of the Fₐ and F_{b} antibody portions, can be directly covalently linked to the gold surface. This novel suggestion is likely to present a simple, high density, and robust means to complex the binding domains of biomarker antibodies with a gold surface.

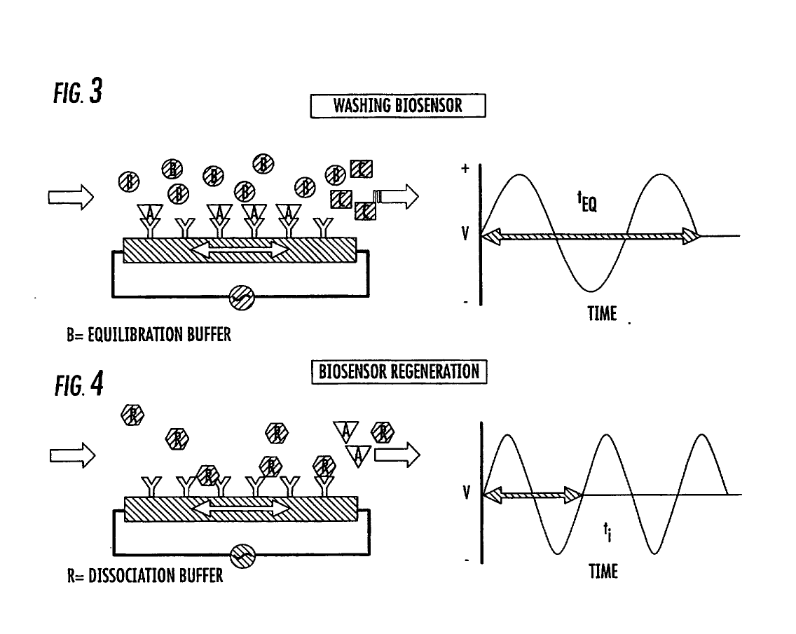

Biomarker/antigen association step (FIGS. 2 and 6): The binding of biomarkers from biofluids to the solid phase detecting agent will occur under favorable conditions with the biofluid alone or by mixing in a flow of “association buffer” such as for example, phosphate buffered saline (PBS) or Tris buffer saline (TBS) with neutral pH 6.8-7.8 range through the biosensor for a defined period of time. By allowing the flowing of an “association buffer” such as, for example, phosphate buffer saline (PBS) or Tris buffer saline (TBS) with neutral pH 6.8-7.8 range through the biosensor, a baseline recording is made. See for example, FIG. 2. Sensor readings can be made as the biofluid is passed across the chip. In the case of a QCM sensor, the rate of change in resonant frequency will be directly proportional to the change in antigen mass flux per unit volume and time (flow), thereby providing a concentration of antigen in the biofluid.

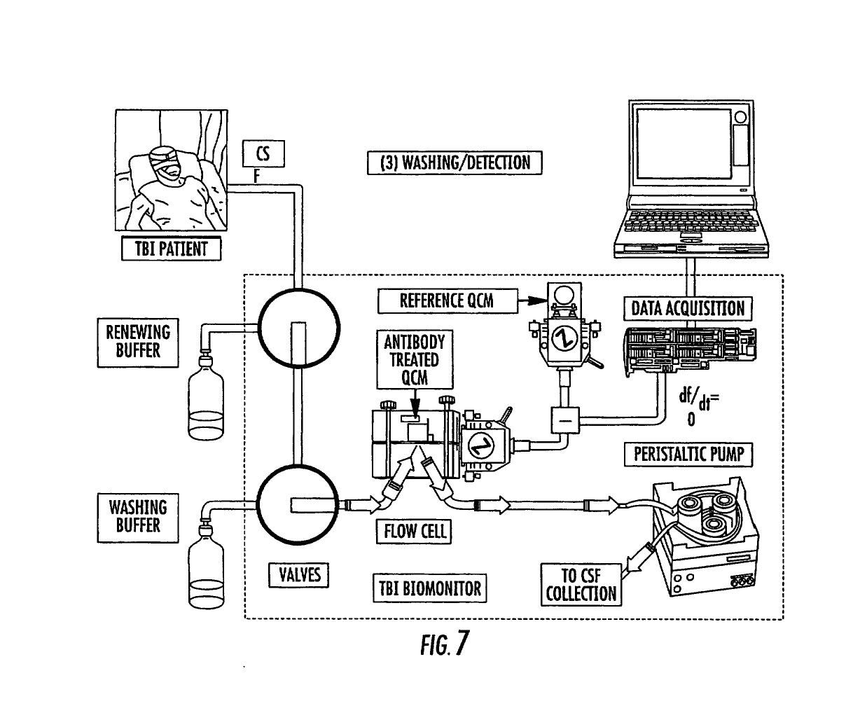

Biosensor washing/detection step (see, for example, FIGS. 3 and 7): Association buffer flow across the biosensor to remove non-specifically bound contaminants. Recording can also be made after equilibrating the biosensor. Measurements will determine the net change in total mass in the QCM from before biofluid introduction to after biofluid introduction. The QCM will respond to mass change (upon addition of biomarker molecules) by shifting its resonant frequency in proportion to the total biomarker mass.

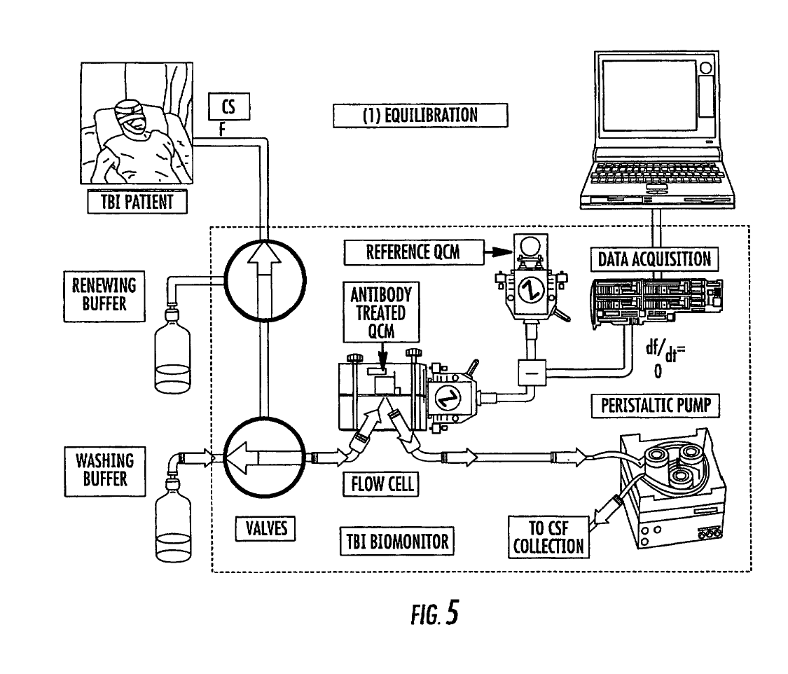

Biosensor regeneration (see, for example, FIG. 8): One of the problems that have hindered real-time/continuous monitoring of patients is that, once binding of biomarkers (antigens) to a detecting agent occurs at neutral pH, there is very little room for new association of newly available biomarkers from the biological fluid flow. The present invention overcomes such limitations, for example, by switching to a dissociation buffer, such as for example 0.1M glycine at pH 2.3. See for example, FIGS. 4 and 8. This allows for the successful real-time monitoring of multiple numbers of biomarkers at a given time and over a given period.

Biosensor re-equilibration steps. Once the biosensor system is regenerated, the system is switched back to association buffer so as to repeat the biomarker measurement cycle (FIGS. 1 and 5). Other steps can be repeated alternately and continuously. The estimated cycle of “association-disassociation” is about 5 minutes, preferably, about 2 minutes, more preferably about 1 minute. Thus, the method is truly a continuous and real-time monitoring of biomarkers endogenous to human/animal disease conditions. This will serve for both diagnosis and monitoring of patient care, especially in the acute phases of disease, when assessment and treatment are most critical. Present methods rely on hours-old data to treat patients at the bedside.

In a preferred embodiment, the QCM surface comprises surface bound capture probes such as, for example, antibodies specific for a biomarker; biomarkers to detect antibodies that may have been generated in vivo due to neural injury; small haptens or molecules arranged in separated, addressable locations, termed herein as “biosites” attached to a solid support. Each biosite comprises specifically-addressable, covalently immobilized small molecules such as haptens, drugs and peptides. These organic capture molecules are designed to have a high affinity association with a specific ligand. The ligand, can optimally be a bispecific ligand. These ligands contain both a domain cognate to the small immobilized organic molecule (capture probe) and cognate to the analyte of interest. The domain cognate to the analyte can associate either directly to this target or to a label on the analyte.

Specific examples of bispecific ligands include, without limitation, antibody:antibody; antibody:receptor; antibody:lectin; receptor:receptor; bispecific anti-bodies; antibody:enzyme; antibody:streptavidin; and antibody:peptide conjugates.

Analytes can include, but are not limited to, dsDNA, ssDNA, total RNA, mRNA, rRNA, peptides, antibodies, proteins, organic enzyme substrates, drugs, and small organic molecules. Preferably, the analytes are neural biomarkers and are indicative of neuronal injury, neuronal disease, neuronal disorders and the like. Examples, include without limitation, neural proteins, peptides, fragments or variants thereof, listed in Table 1.

Preferably, antibodies specific for neural biomarkers are immobilized at high density on the appropriate surface whilst still maintaining their functional configuration and preventing stearic hindrance of the binding sites. An example is the use of self-assembling long chain alkyl membrane systems (SAMSs) on glass or silica and gold surfaces. The terminal functional groups on each chain are designed to react with specific groups on antibodies or antibody fractions to form a uniform geometrical array of antigen binding sites. Another method is to use a SAM formed from a mixture of two long chain alkane thiolates, one with a terminal functional group for reaction with, for example, Fab-SH groups and the other presenting a short oligomer of ethylene glycol to resist the non-specific adsorption of protein at the membrane surface. This mixture would allow the possibility of controlling the spacing of the covalently bound antibody fraction and optimizing specific antigen binding. Other suitable materials for immobilizing antibodies, peptides, etc, onto the quartz crystal include, without limitation, polyethyleneimine, aminopropyl-tri-ethoxysilane, protein A, polyacrylamide, and avidin.

Most immunological reactions have large association constants (Kₐ's of 10⁵-10⁹ M⁻¹). The Kₐ's are composed of large forward [k₁] and small reverse [k₋₁] rate constants ranging from 10⁷ to 10⁹ M⁻¹ s⁻¹ and 10² to 10⁻⁴ s⁻¹ respectively. Preferred antibodies include antibodies with sufficiently fast antigen dissociation rates to allow reversible measurements in real time for continuous and sequential measurements of the antigen without the need to replace the antibody or reverse the binding by the use of chaotropic solutions. Examples include, but not limited to catalytic antibodies; haptens designed to mimic the stereoelectronic features of transition states.

In another aspect of the invention, the capture probe may comprise nucleic acid molecules. Single stranded-DNA (ssDNA) can be grown on the surface of optical fibers and detection of specific binding is accomplished via the hybridization process with complementary ssDNA in a sample by using the fluorescence of ethidium bromide trapped in the double-stranded regions of the bound DNA. The probe can be repeatedly regenerated for further use by a short immersion in hot buffer. Detection of hybridization may be further improved by covalently immobilizing the double stranded DNA (dsDNA) sensitive fluorescent dye directly onto the immobilized ssDNA at the glass fiber surface.

Conversely, if the user so desires, the format for a QCM can be inverted so that the macromolecular ligand becomes the capture probe. Thus, a QCM may comprise large macromolecules such as, without limitation, antibodies, proteins, polysaccharides, peptides, or receptors as the immobilized capture probe. See for example, proteins listed in Table 1. In turn, unique small molecule tags having a specific, high affinity association for the macromolecular biosites are covalently attached to various probes cognate to the analyte. These labeled probes now represent the bispecific component cognate to both the capture macromolecule and the target analyte.

In another preferred embodiment, the sensor in the system is made to oscillate. The sensor can be made to oscillate in a number of ways, e.g. by the use of surface acoustic wave devices, resonance quartz crystal devices, acoustic plate mode and thin membrane flexural plate devices. Many different sensors, suitable for use in the invention, are available from commercial sources. The sensor can be a surface acoustic wave device, however, a quartz crystal microbalance (QCM) is preferred. The QCM is typically a disc of crystalline quartz with gold electrodes on the top and lower surfaces. It undergoes a shearing oscillation when an alternating voltage is applied to the electrodes, due to the converse piezo-electric effect. Increasing the voltage increases the amplitude of oscillation of the, QCM.

The present invention may be used to study any molecular interaction, but is particularly suitable for the study of antibody/antigen interactions and receptor/ligand interactions, enzyme/ligand interactions, or an interaction between a large macromolecule and its natural binding partner. The method may also be applied to the study of hybridization events between polynucleotides, e.g. a biomarker can be a nucleic acid molecule of any polypeptide listed in Table 1. Thus, in the first aspect of the invention, the ligand may be, for example, a protein, an antibody or antigen, an enzyme, an enzyme inhibitor, a polynucleotide or a large macromolecule such as a large plasmid or virus. Either material may be bound to the surface or particle.

In a preferred embodiment, detection of one or more neural biomarkers is diagnostic of neural damage and/or neuronal disease. Preferably, neural biomarker detection is in real-time.

Examples of neural biomarkers, include but are not limited to: neural proteins, such as for example, axonal proteins—NF-200 (NF-H), NF-160 (NF-M), NF-68 (NF-L); amyloid precursor protein; dendritic proteins—alpha-tubulin (P02551), beta-tubulin (P0 4691), MAP-2A/B, MAP-2C, Tau, Dynamin-1 (P21575), Dynactin (Q13561), P24; somal proteins—UCH-L1 (Q00981), PEBP (P31044), NSE (P07323), Thy 1.1, Prion, Huntington; presynaptic proteins—synapsin-1, synapsin-2, alpha-synuclein (p37377), beta-synuclein (Q63754), GAP43, synaptophysin, synaptotagmin (P21707), syntaxin; post-synaptic proteins —PSD95, PSD93, NMDA-receptor (including all subtypes); demyelination biomarkers—myelin basic protein (MBP), myelin proteolipid protein; glial proteins—GFAP (P47819), protein disulfide isomerase (PDI-P04785); neurotransmitter biomarkers—cholinergic biomarkers: acetylcholine esterase, choline acetyltransferase; dopaminergic biomarkers—tyrosine hydroxylase (TH), phospho-TH, DARPP32; noradrenergic biomarkers—dopamine beta-hydroxylase (DbH); serotonergic biomarkers—tryptophan hydroxylase (TrH); glutamatergic biomarkers—glutaminase, glutamine synthetase; GABAergic biomarkers—GABA transaminase (4-aminobutyrate-2-ketoglutarate transaminase [GABAT]), glutamic acid decarboxylase (GAD25, 44, 65, 67); neurotransmitter receptors—beta-adrenoreceptor subtypes, (e.g. beta (2)), alpha-adrenoreceptor subtypes, (e.g. (alpha (2c)), GABA receptors (e.g. GABA(B)), metabotropic glutamate receptor (e.g. mGluR3), NMDA receptor subunits (e.g. NR1A2B), Glutamate receptor subunits (e.g. GluR4), 5-HT serotonin receptors (e.g. 5-HT(3)), dopamine receptors (e.g. D4), muscarinic Ach receptors (e.g. M1), nicotinic acetylcholine receptor (e.g. alpha-7); neurotransmitter transporters—norepinephrine transporter (NET), dopamine transporter (DAT), serotonin transporter (SERT), vesicular transporter proteins (VMAT1 and VMAT2), GABA transporter vesicular inhibitory amino acid transporter (VIAAT/VGAT), glutamate transporter (e.g. GLT1), vesicular acetylcholine transporter, choline transporter (e.g. CHT1); other protein biomarkers include, but not limited to vimentin (P31000), CK-BB (P07335), 14-3-3-epsilon (P42655), MMP2, MMP9.

Without wishing to be bound by theory, upon injury, structural and functional integrity of the cell membrane and blood brain barrier are compromised. Brain-specific and brain-enriched proteins are released into the extracellular space and subsequently into the CSF and blood.

In a preferred embodiment, detection of at least one neural protein in CSF, blond, or other biological fluids, is diagnostic of the severity of brain injury and/or the monitoring of the progression of therapy. Preferably, the neural proteins are detected during the early stages of injury. An increase in the amount of neural proteins, fragments or derivatives thereof, in a patient suffering from a neural injury, neuronal disorder as compared to a normal healthy individual, will be diagnostic of a neural injury and/or neuronal disorder. Any animal that expresses the neural proteins, such as for example, those listed in Table 1, can be used as a subject from which a biological sample is obtained. Preferably, the subject is a mammal, such as for example, a human, dog, cat, horse, cow, pig, sheep, goat, chicken, primate, rat, or mouse. More preferably, the subject is a human. Particularly preferred are subjects suspected of having or at risk for developing traumatic or non-traumatic nervous system injuries, such as victims of brain injury caused by traumatic insults (e.g. gunshots wounds, automobile accidents, sports accidents, shaken baby syndrome), ischemic events (e.g. stroke, cerebral hemorrhage, cardiac arrest), spinal cord injury, neurodegenerative disorders (such as Alzheimer's, Huntington's, and Parkinson's diseases; Prion-related disease; other forms of dementia, and spinal cord degeneration), epilepsy, substance abuse (e.g., from amphetamines, methamphetamine/Speed, Ecstasy/MDMA, or ethanol and cocaine), and peripheral nervous system pathologies such as diabetic neuropathy, chemotherapy-induced neuropathy and neuropathic pain, peripheral nerve damage or atrophy (ALS), multiple sclerosis (MS).

In another preferred embodiment, detection of at least one neural protein in CSF, blood, or other biological fluids, is diagnostic of the severity of injury following a variety of CNS insults, such as for example, stroke, spinal cord injury, or neurotoxicity caused by alcohol or substance abuse (e.g. ecstasy, methamphetamine, etc.)

In another preferred embodiment, at least one neural biomarker is detected in real-time. Preferably, about two biomarkers are detected in real-time, more preferably, about four biomarkers are detected in real-time; more preferably, about eight biomarkers are detected in real-time; more preferably up to twenty biomarkers are detected in real-time. The CNS comprises many brain-specific and brain-enriched proteins that are preferable biomarkers in the diagnosis of brain injury, neural injury, neural disorders and the like. For example, the neural specific biomarkers can include axonal proteins such as neurofilament-heavy (NF-200), neurofilament-medium (NF-160), neurofilament-light (NF-68), and amyloid precursor protein; dendritic proteins such as alpha-tubulin, beta-tubulin, MAP-2A/B/C. tau, dynamin-1, dynactin; and proteins found in the soma (cell body) including ubiquitin C-terminal hydrolase L1 (U CH-L1), PEBP, neuronal-specific enolase (NSE), NeuN, Thy 1.1, Prion and Huntington. There are also proteins found pre-synaptically and post-synaptically. Moreover, different types of neurons exhibit distinct neurotransmitter-specific enzyme pathway proteins. For example, acetylcholine esterase is found only in cholinergic neurons while tyrosine hydroxylase (TH) is exclusive to dopaminergic neurons. Other neurotransmitter-specific enzyme pathway proteins include dopamine beta hydroxylase (DbH) in noradrenergic neurons, tryptophan hydroxylase (TrH) in serotonergic neurons, glutaminase and glutamine synthetase in glutamatergic neurons, and GABA transaminase and glutamic acid decarboxylase in GABAergic neurons. Furthermore, proteins such as GFAP and protein disulfide isomerase (PDI) are only synthesized in glial cells of the CNS, a feature that could be exploited to further understand the extent of damage to the CNS.

In another preferred embodiment, the invention provides for the quantitative detection of damage to the CNS, PNS and/or brain injury at a subcellular level. Depending on the type and severity of injury, neurons can undergo damage in specific cellular regions. Preferably, at least one biomarker is detected in real-time which is indicative of the quantitative damage of neural injury, more preferably, about two biomarkers are detected in real-time which are indicative of the quantitative damage of neural injury, more preferably, about four biomarkers are detected in real-time which are indicative of the quantitative damage of neural injury, more preferably, about eight biomarkers are indicative of the quantitative damage of neural injury, more preferably up to twenty biomarkers are indicative of the quantitative damage of neural injury. For example, detection of certain biomarkers, such as for example, axonal proteins, fragments and derivatives thereof include, but not limited to: NF-200 (NF-H), NF-160 (NF-M), NF-68 (NF-L), and the like, differentiates between axonal versus dendritic damage. Non-limiting examples of dendritic proteins, peptides, fragments and derivatives thereof, include, but not limited to: alpha-tubulin (P02551), beta-tubulin (P04691), MAP-2A/B, MAP-2C, Tau, Dynamin-1 (P21575), Dynactin (Q13561), p24 (neural-specific MAP). Furthermore, detection of different biomarkers not only differentiate between, for example, axonal or dendritic damage, but allow for the assessment of synaptic pathology, specific injury to elements of the pre-synaptic terminal and post-synaptic density.

In another preferred embodiment, detection of certain biomarkers are diagnostic of the specific cell type affected following injury since neurons and glia possess distinct proteins. Preferably, at least one cell type specific biomarker is detected in real-time, more preferably about two cell type specific biomarkers are detected in real-time; more preferably, about four cell type specific biomarkers are detected in real-time; more preferably, about eight cell type specific biomarkers are detected in real-time; more preferably, up to about twenty cell type specific biomarkers are detected in real-time. For example, detection of glial proteins, peptides, fragments and derivatives thereof is diagnostic of glial cell damage. Examples of glial proteins, include, but not limited to: GFAP (P47819), protein disulfide isomerase (PDI-P04785).

The ability to detect and monitor levels of these proteins after CNS injury provides enhanced diagnostic capability by allowing clinicians (1) to determine the level of injury severity in patients with various CNS injuries, (2) to monitor patients for signs of secondary CNS injuries that may elicit these cellular changes and (3) to monitor the effects of therapy by examination of these proteins in CSF or blood. Unlike other organ-based diseases where rapid diagnostics for surrogate biomarkers prove invaluable to the course of action taken to treat the disease, no such rapid, definitive diagnostic tests exist for traumatic or ischemic brain injury that might provide physicians with quantifiable neurochemical markers to help determine the seriousness of the injury, the anatomical and cellular pathology of the injury, and the implementation of appropriate medical management and treatment.

In an illustrative example, not meant to limit or construe the invention in any way, identification of which brain-specific and brain-enriched proteins are elevated in CSF following traumatic brain injury (TBI) is diagnostic, for example, of brain injury, the degree of brain injury, type of cellular damage and degree of cellular damage. Furthermore, detection of certain brain-specific and brain-enriched proteins, fragments and derivatives thereof, is diagnostic of the type and degree of cellular damage. For example, increased levels of a variety of brain-specific and brain-enriched proteins in the CSF 48 hours following injury, were detected. Specifically, elevated levels of the somal protein ubiquitin C-terminal hydrolase L1 (UCH-L1) the dendritic protein p24, and α-synuclein, a pre-synaptic protein were detected following injury.

In a preferred embodiment, biomarkers indicative of CNS, PNS and/or brain injury, the degree injury, type of cellular damage and degree of cellular damage are detected. Preferably, antibodies specific for such biomarkers are adsorbed onto the QCM detection surface. Detection in changes of mass are indicative of the specific biomarker and the amount of biomarker detected, i.e. μg/ml.

In another preferred embodiment, detection of certain brain-specific and brain-enriched proteins, fragments and derivatives thereof, is diagnostic of the type and degree of cellular damage. For example, increased levels of a variety of brain-specific and brain-enriched proteins in the CSF 48 hours following injury, were detected. Specifically, elevated levels of the somal protein ubiquitin C-terminal hydrolase L1 (UCH-L1) the dendritic protein p24, and α-synuclein, a pre-synaptic protein were detected following injury. Therefore, detection of one or more of such types of biomarkers are indicative of the type and degree of cellular damage. As the amounts and/or types of biomarkers in a sample change as measured by the QCM over periods of time, the severity, location, type of neural damage, can be monitored. Preferably, changes in mass of biomarkers is detected within about 10 minutes for each new sample, more preferably, changes in mass of biomarkers is detected within about 5 minutes for each new sample; more preferably, changes in mass of biomarkers is detected within about two minutes for each new sample; more preferably changes in mass of biomarkers is detected within about 1 minute for each new sample.

Examples of biomarkers are illustrated in Table 1. These are illustrative examples and are not meant to limit or construe the invention in any way. Peptides, fragments and variants thereof are within the scope of the invention.

TABLE 1
Examples of Neural Proteins as Biomarkers for Nervous
System Injury and Other Nervous System Disorders

Neural Subcellular Protein Biomarkers

Axonal Proteins

NF-200 (NF-H)
NF-160 (NF-M)
NF-68 (NF-L)
Amyloid precursor protein

Dendritic Proteins

alpha-Tubulin (P02551)
beta-Tubulin (P04691)
MAP-2A/B
MAP-2C
Tau
Dynamin-1 (P21575)
Phocein
Dynactin (Q13561)
p24 microtubule-associated protein
Vimentin (P31000)

Somal Proteins

UCH-L1 (Q00981)
PEBP (P31044)
NSE (P07323)
CK-BB (P07335)
Thy 1.1
Prion protein
Huntingtin
14-3-3 proteins (e.g. 14-3-3-epsolon (P42655))

Neural nuclear proteins

S/G(2) nuclear autoantigen (SG2NA)
NeuN

Presynaptic Proteins

Synapsin-1
Synapsin-2
alpha-Synuclein (P37377)
beta-Synuclein (Q63754)
GAP43
Synaptophysin
Synaptotagmin (P21707)
Syntaxin

Post-Synaptic Proteins

PSD95
PSD93
NMDA-receptor (and all subtypes)
AMPA-kainate receptor (all subtypes)

Neuronal subtype Biomarkers
Neuronal subtypes-Neurotransmitter Receptors
beta-adrenoceptor subtypes (e.g. beta(2))
Alpha-adrenoceptors subtypes (e.g. alpha(2c))
GABA receptors (e.g. GABA(B))
Metabotropic glutamate receptor (e.g. mGluR3)
NMDA receptor subunits (e.g. NR1A2B)
Glutamate receptor subunits (AMPA, Kainate receptors
(e.g. GluR4. GluR1)
5-HT serotonin receptors (e.g. 5-HT(3))
Dopamine receptors (e.g. D4)
Muscarinic Ach receptors (e.g. M1)
Nicotinic Acetylcholine Receptor (e.g. alpha-7)
Nervous System Anatomical biomarkers
(CNS + PNS)
Hippocampus

SCG10, Stathmin

Cerebellum

Purkinje cell protein-2 (Pcp2)

Cereborcoretx

H-2Z1 gene product

Thalamus

CD15 (3-fucosyl-N-acetyl-lactosamine) epitope

Hypothalamus

Orexin receptors (OX-1R and OX-2R)- appetite
Orexins (hypothalamus-specific peptides)

CORPUS CALLOSUM

MBP, MOG, PLP

Spinal Cord

Schwann cell myelin protein

Striatum

Rhes (Ras homolog enriched in striatum)
Striatin

Peripheral ganglia

Gadd45a

Peripherial nerve fiber(sensory + motor)

Peripherin

Myelin-Oligodendrocyte Biomarkers

Myelin basic protein (MBP)
Myelin proteolipid protein (PLP)
Myelin Oligodendrocyte specific protein (MOSP)
Oligodendrocyte NS-1 protein
Myelin Oligodendrocyte glycoprotein (MOG)

Glial Protein Biomarkers

GFAP (P47819)
Protein disulfide isomerase (PDI) - P04785

Microglia protein Biomarkers

PTPase (CD45)
CD40
MHC class II antigens
CD11b

Phocein
Schwann cell markers

Schwann cell myelin protein

Neural Stem Biomarkers (Neural stem cells, neural progenitor
cells, neuroblasts, glioblasts, early-differentiated neurons

Beta-III tubulin
Alpha-internexin
HuC, HuD
MAP1b
TUC-4
OX-42
Osteopontin

Neuronal subtype Biomarkers
Neuronal subtypes-Neurotransmitter Transporters

Norepinephrine Transporter (NET)
Dopamine transporter (DAT)
Serotonin transporter (SERT)
Vesicular transporter proteins (VMAT1 and VMAT2)
GABA transporter vesicular inhibitory amino acid transporter
(VIAAT/VGAT)
Glutamate Transporter (e.g. GLT1)
Vesicular acetylcholine transporter
Vesicular Glutamate Transporter 1
[VGLUT1; BNPI] and VGLUT2
Choline transporter, (e.g. CHT1)

Neuronal subtype Biomarkers: Neurotransmitter-Synthesis-
Metabolism

Cholinergic Biomarkers

Acetylcholine Esterase
Choline acetyltransferase [ChAT]

Dopaminergic Biomarkers

Tyrosine Hydroxylase (TH)
Phospho-TH
DARPP32

Noradrenergic Biomarkers

Dopamine beta-hydroxylase (DbH)

Adrenergic Biomarkers

Phenylethanolamine N-methyltransferase (PNMT)

Serotonergic Biomarkers

Tryptophan Hydroxylase (TrH)

Glutamatergic Biomarkers

Glutaminase
Glutamine synthetase

GABAergic Biomarkers

GABA transaminase [GABAT])
DARPP32

Other Neuron-specific proteins
PH8 (S Serotonergic Dopaminergic
PEP-19, a neuron-specific protein
Neurocalcin (NC),
a neuron-specific EF-hand Ca2+-binding protein
Encephalopsin

As described above, the invention provides the step of correlating the presence or amount of one or more neural protein(s) with the severity and/or type of nerve cell injury. The amount of a neural proteins, peptides, fragments, derivatives or the modified forms, thereof, directly.

The invention is not limited to samples taken from a patient at certain times but also provides for the continuous monitoring of changes in the mass of biomarkers from a continual flow of sample. For example, FIGS. 5 and 6 are illustrative of the real-time detection of biomarkers in a continuous flow of sample from a patient. The flow of the sample can be regulated so that maximal detection of biomarkers is achieved. Flow rates can vary depending on the extent of neural injury as the operator may adjust the flow to optimize detection of biomarkers. For example, where injury is extensive and the concentration of biomarkers is high, the operator may adjust the flow rate to allow for binding of biomarkers that are present in lower concentrations as compared to a biomarker that can be many fold higher in concentration in the sample. For illustrative purposes, a biomarker designated as biomarker “1” may be present at a higher concentration than biomarkers “2”, “3”, “4” or “5” due to the type or severity of injury, and the operator may adjust the flow rate to allow for the binding and detection of biomarkers 2, 3, 4 or 5. Or conversely, as the concentration of biomarker “1” decreases, and biomarker “2” and “5” increases the flow rate can be adjusted to allow for detection and binding of biomarkers “3” and “4”.

As an illustrative example, not meant to limit or construe the invention in any way, the following is provided. The QCM is treated with a capture probe which adsorbs to the surface of the QCM. The QCM is equilibrated with renewing buffer and/or a washing buffer to remove any material not bound to the QCM surface. The reference QCM is set-up to provide a baseline. Appropriate controls are also adsorbed onto the QCM to eliminate any non-specific binding. A normal peristaltic pump is used to control the flow rate. A catheter needle is inserted into a patient, for example, intravenously, spinal tap and the like. Sample flows from the patient at a regulated flow rate, similar to flow rates of intravenous drips, patient is The sample from a patient enters a flow cell which also controls the volume of sample and flow rate of sample leading to the QCM. Changes in mass are detected and compared to the changes in mass with the reference QCM. Data is collected and analyzed by an appropriate algorithm. Sample that has passed through the QCM is collected and stored, if desired, for future reference.

The system is preferably, re-used for detection of other biomarkers present in a patient sample. Prior to a new a sample, the QCM crystal can be subjected to a regeneration step. The entire system is washed with washing buffer and bound markers are eluted and the system is washed again. In a preferred embodiment, the regeneration of the system is about 5 minutes, more preferably, the regeneration of the system is about 3 minutes, more preferably, the regeneration of the system is about 2 minutes, more preferably, the regeneration of the system is about thirty seconds.

Once the system has been flushed with the renewing buffer and the washing buffer, the patient sample continues to flow. The sample can be processed by the QCM at time intervals desired by the operator. For example, if the injury appears to be life-threatening, then samples are taken at shorter time intervals to maximize the information, for example, to diagnose whether there has been severe damage, the type of damage, the location of the damage and the progression of the injury. This will provide a medical practitioner to make split-second decisions as to the medical procedures that need to be made to avoid further injury and to stabilize the patients condition. If, however, the patient is stabilized the time interval between different samples monitored may be longer.

Molecules that are specific for different biomarkers are adsorbed to the surface of the crystal in a defined address or site. By “site” is meant the biological molecules or capture probes that are deposited on the top surface of the reaction substrate, or base material. Under appropriate conditions, an association or hybridization can occur between the capture probe and a target molecule. The component strands of the biological molecule form the site since there is the potential of a reaction occurring between each component strand of the biological molecule and the target molecule. The maximum number of sites per reaction chamber will depend on the size of the biosensor and on the practical optical resolution of the accompanying detector/imager. For example, an array of 16 (4×4 array) sites may be deposited on the hybridization substrate or base material that eventually forms the bottom of the entire reaction vessel. Each site comprises a circle of approximately 25-200 microns (μm) in diameter. Thus, for a 16 site array, each of the 16×200 μm diameter area contains a uniform field of probes attached to the hybridization substrate (base material) in a concentration which is highly dependent on the probe size and the well size. Each 25-200 μm diameter area can contain millions of probe molecules. Also, each of the 16 different sites (probe sites) can contain one type of probe. Thus, 16 different probe types can be assayed in an array containing 16 sites (4×4 array) per biosensor. As another example, four separate 10×10 arrays (400 sites) can be generated to fit into one defined area with sufficient spacing between each of the 400 sites. For this 10×10 format, 400 hybridization experiments are possible within a single reaction chamber corresponding to 38,400 (96×400) assays/hybridization that can be performed nearly simultaneously.

In another aspect, the invention provides methods for aiding a diagnosis for neural injury and/or neural disorder using one or more markers, for example markers listed in Table 1, fragments, peptides, derivatives and variants thereof. These markers can be used alone, in combination with other markers in any set, or with entirely different markers in aiding neural injury and/or neural disorder diagnosis. The markers are differentially present in samples of a patient with, for example neural injury, and a normal subject in whom neural injury is undetectable. For example, some of the markers are expressed at an elevated level and/or are present at a higher frequency in patients with neural injury and/or neural disorder than in normal subjects. Therefore, detection of one or more of these markers in a person would provide useful information regarding the probability, type, degree, severity and location that the person may have neural injury.

Accordingly, embodiments of the invention include methods for aiding a diagnosis of neural injury and/or neural disorder, wherein the method comprises: (a) detecting at least one marker in a sample, wherein the marker is selected from Table 1; and (b) correlating the detection of the marker or markers with a probable diagnosis of neural injury and/or neural disorder in real-time. The correlation may take into account the amount of the marker or markers in the sample compared to a control amount of the marker or markers (up or down regulation of the marker or markers) (e.g., in normal subjects in whom neural injury and/or neural disorder is undetectable). The correlation may take into account the presence or absence of the markers in a test sample and the frequency of detection of the same markers in a control. The correlation may take into account both of such factors to facilitate determination of whether a subject is suffering from neural injury and/or neural disorder or not.

Using the methods and apparatus disclosed herein, one or more markers can be detected in real-time. Preferably, a sample is tested for the presence of a plurality of markers. Detecting the presence of a plurality of markers, rather than a single marker alone, would provide more information for the diagnostician. Specifically, the detection of a plurality of markers in a sample would increase the percentage of true positive and true negative diagnoses and would decrease the percentage of false positive or false negative diagnoses.

The detection of the marker or markers is then correlated with a diagnosis of type of neural injury and/or neural disorder, the degree and severity of the neural injury and/or disorder, location of neural injury. In some embodiments, the detection of the mere presence or absence of a marker, without quantifying the amount of marker, is useful and can be correlated with a diagnosis of neural injury and/or neural disorder.

When the markers are quantified, they can be compared to a control. A control can be, e.g., the average or median amount of marker present in comparable samples of normal subjects in whom neural injury and/or neural disorder is undetectable. The control amount is measured under the same or substantially similar experimental conditions as in measuring the test amount. For example, if a test sample is obtained from a subject's blood serum sample and a marker is detected using a particular probe, then a control amount of the marker is preferably determined from a serum sample of a patient using the same probe. It is preferred that the control amount of marker is determined based upon a significant number of samples from normal subjects who do not have neural injury and/or neural disorder so that it reflects variations of the marker amounts in that population.

Data generated by apparatus of the invention can then be analyzed by a computer software. The software can comprise code that converts signal from the QCM into computer readable form. The software also can include code that applies an algorithm to the analysis of the signal to determine whether the signal represents a “peak” in the signal corresponding to a marker of this invention, or other useful markers. The software also can include code that executes an algorithm that compares signal from a test sample to a typical signal characteristic of “normal” and neural injury and/or neural disorder and determines the closeness of fit between the two signals. The software also can include code indicating which the test sample is closest to, thereby providing a diagnosis of type, severity, degree and location of neural injury.

### Data Acquisition and Processing.

Data generated by desorption and detection of markers can be analyzed using any suitable means. In one embodiment, data is analyzed with the use of a programmable digital computer. The computer program generally contains a readable medium that stores codes. Certain code can be devoted to memory that includes the location of each feature on a probe, the identity of the adsorbent at that feature and the elution conditions used to wash the adsorbent. The computer also contains code that receives as input, data on the strength of the signal at various molecular masses received from a particular addressable location on the probe. This data can indicate the number of markers detected, including the strength of the signal generated by each marker.

Data analysis can include the steps of recording shifts in frequency verses time, or over a fixed period of time, and correlating this to either an absolute analyte value (with calibration) or to a relative value (increase/decrease over time).

The computer can transform the resulting data into various formats for displaying. In one format, referred to as “spectrum view or retentate map,” a standard spectral view can be displayed, wherein the view depicts the quantity of marker reaching the detector at each particular molecular weight. In another format, referred to as “peak map,” only the peak height and mass information are retained from the spectrum view, yielding a cleaner image and enabling markers with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique markers and markers which are up- or down-regulated between samples. Marker profiles (spectra) from any two samples may be compared visually. In yet another format, Spotfire Scatter Plot can be used, wherein markers that are detected are plotted as a dot in a plot, wherein one axis of the plot represents the apparent molecular of the markers detected and another axis represents the signal intensity of markers detected. For each sample, markers that are detected and the amount of markers present in the sample can be saved in a computer readable medium. This data can then be compared to a control (e.g., a profile or quantity of markers detected in control, e.g., individuals in whom neural injury and/or neural disorder is undetectable).

In a preferred embodiment, the software uses the LabView programming environment. The software controls actuation and timing of the fluid selection valve. The software interfaces with the frequency counters to collect the oscillation frequency. The initial frequency at the beginning of acquisition is recorded, with the difference in frequency calculated in respect to the initial value. The frequency difference is plotted against time and recorded to a data file. The software also interfaces with the National Instruments analog-to-digital converter and records the oscillation amplitude output by the RF oscillator circuit. The initial amplitude at the beginning of acquisition is recorded, with the difference in amplitude calculated with respect to the initial value. The amplitude difference is plotted against time and recorded to a data file.

### Methods for Preparing Antibodies

In another aspect, the present invention contemplates an antibody that is immunoreactive with a polypeptide. Reference to antibodies throughout the specification includes whole polyclonal and monoclonal antibodies (mAbs), and parts thereof, either alone or conjugated with other moieties. Antibody parts include Fab and F(ab)₂ fragments and single chain antibodies. The antibodies may be made in vivo in suitable laboratory animals or in vitro using recombinant DNA techniques. In a preferred embodiment, an antibody is a polyclonal antibody.

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a rabbit, a mouse, a rat, a hamster or a guinea pig. Because of the relatively large blood volume of rabbits, a rabbit is a preferred choice for production of polyclonal antibodies.

Antibodies, both polyclonal and monoclonal, specific for given polypeptides may be prepared using conventional immunization techniques, as will be generally known to those of skill in the art. A composition comprising antigenic epitopes of particular polypeptides can be used to immunize one or more experimental animals, such as a rabbit or mouse, which will then proceed to produce specific antibodies against the polypeptide. Polyclonal antisera may be obtained, after allowing time for antibody generation, simply by bleeding the animal and preparing serum samples from the whole blood.

The amount of immunogen composition used in the production of polyclonal antibodies varies upon the nature of the immunogen, as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored or the animal can be used to generate mAbs (below), or both.

One of the important features provided by the present invention is a polyclonal sera that is relatively homogenous with respect to the specificity of the antibodies therein. Typically, polyclonal antisera is derived from a variety of different “clones,” i.e. B-cells of different lineage. mAbs, by contrast, are defined as coming from antibody-producing cells with a common B-cell ancestor, hence their “mono” clonality.

Polyclonal antisera according to present invention is produced against peptides that are predicted to comprise whole, intact epitopes, see for example, Table 1. It is believed that these epitopes are therefore more stable in an immunologic sense and thus empress a more consistent immunologic target for the immune system. Under this model, the number of potential B-cell clones that will respond to this peptide is considerably smaller and, hence, the homogeneity of the resulting sera will be higher. In various embodiments, the present invention provides for polyclonal antisera where the clonality, i.e., the percentage of clone reacting with the same molecular determinant, is at least 80%. Even higher clonality up to 90% or 95% or greater is contemplated.

To obtain mAbs, one would also initially immunize an experimental animal, often preferably a mouse, with a polypeptide-containing composition, see for example Table 1. After a period of time sufficient to allow antibody generation, one would obtain a population of spleen or lymph cells from the animal. The spleen or lymph cells can then be fused with cell lines, such as human or mouse myeloma strains, to produce antibody-secreting hybridomas. These hybridomas may be isolated to obtain individual clones which can then be screened for production of antibody to the desired polypeptide. Following immunization, spleen cells are removed and fused, using a standard fusion protocol with plasmacytoma cells to produce hybridomas secreting mAbs against a polypeptide of interest. Hybridomas which produce mAbs to the selected antigens are identified using standard techniques, such as ELISA and Western blot methods. Hybridoma clones can then be cultured in liquid media and the culture supernatants purified to provide the polypeptide of interest-specific mAbs.

Of particular utility to the present invention are antibodies tagged with a fluorescent or enzymatic molecule. Methods of tagging antibodies are well known to those of skill in the art and a large number of such antibodies are available commercially Fluorescent tags include, but are not limited to, fluorescein, phycoerythrin, and Texas red. Enzymatic tags, include, but are not limited to, alkaline phosphatase and horseradish peroxidase.

In addition to methods by which a receptor or molecule of interest is immobilized and used to bind an analyte, general methods exist for immobilizing members of a class of reactants. For example, protein A or protein G may be immobilized and used subsequently to bind specific immunoglobulins, which in turn will bind specific analytes. A more general approach is built around the strong and specific reaction between other ligands and receptors such as avidin and biotin. Avidin may be immobilized on a solid support or attached to a gel and used to bind antibodies or other reactants to which biotin has been linked covalently. That allows the production of surfaces to which a variety of reactants can be attached readily and quickly (see Savage et al., Avidin-Biotin Chemistry: A Handbook. Pierce Chemical Company, 1992).

### Biosensors For Chemical And Biological Agents

In accordance with the invention, other applications include monitoring of pathogen, pathogenic contamination in water/fluid supplies as a result of bioterrorism or accidental human activity or natural epidemics. A similar situation will apply to pathogen contamination in water supplies where water samples can be monitored as frequently as desired. Thus the apparatus of the present invention is useful for a wide range of applications.

For example, if the biosensor is used as a detector of chemical agents, the particular coating chosen for the crystal substrate should preferably readily adsorb the molecules of the analyte, to provide fast response times and a high degree of sensitivity to the analyte over a broad temperature range, but do so without damping the generated waves. An example, is a fluoropolymer coating. The fluoropolymer may be a copolymer comprising perfluoro-2,2-dimethyl-1,3-dioxole. The co-monomer typically is fluorinated. Useful fluoropolymers are disclosed in U.S. Pat. Nos. 4,754,009 and 5,000,547, the disclosures of which are incorporated herein by reference.

In accordance with the present invention, the quartz crystal may be coated with an adsorbent material suitable for adsorbing a first molecule. However, the first molecule can be directly adsorbed onto the crystal surface. The adsorbent material or coating may be applied to the crystal substrate and electrodes using the following procedure. The crystal substrate and electrodes are first cleaned using acetone and methanol. The adsorbent material is dissolved in a suitable organic solvent if the adsorbent material is a solid, for example polytetrafluoroethylene (TEFLON™) is dissolved in a fluorinated hydrocarbon solvent to produce a solution having a concentration of between about 1-6% TEFLON™, by weight. The concentration of polytetrafluoroethylene (TEFLON™) in the solution is related to the desired coating thickness. The more concentrated the solution, the thicker the resulting coating will be. About 7-10 drops of the solution is then applied to the substrate and electrodes to completely cover one side of the sensor. The coated substrate is then placed on a spin coater, a machine adapted to rotate at variable speed, with the preferred speed range being about 500-6000 RPM, for a duration of about two minutes. The selected spin rate depends on the targeted coating thickness, with higher spin rates being selected for thinner coatings. After spin coating, the sensor is air dried for approximately one minute, with the aforementioned steps then being repeated for each side of the sensor. The sensor can then be cured, if desired, at a temperature of 100° C. for about two hours. Alternatively, if the coating is being applied to surface acoustic wave sensors or thin film resonator sensors, spray-coating and dip-coating techniques may be employed, respectively.

In one aspect, the biomonitor can be used for pathogen contamination detection, water/fluid supplies are used, for example, pathogen contamination of water supplies. Other applications, include, but are not limited to, bioterrorism or accidental human activity or natural epidemic control or monitoring, a specific panel of pathogens to include all biological microorganisms that express proteins on their outer surface, can be immunologically and sensitively detected in real-time. The device is a diagnostic tool providing both instantaneous readings and a historical record for prompting appropriate action.

### Alternative Detection Methods.

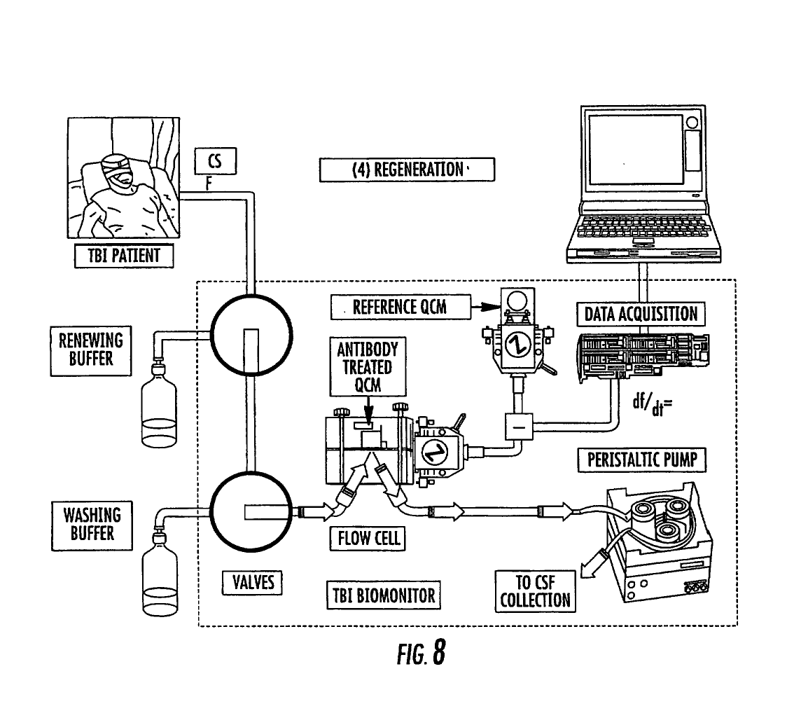

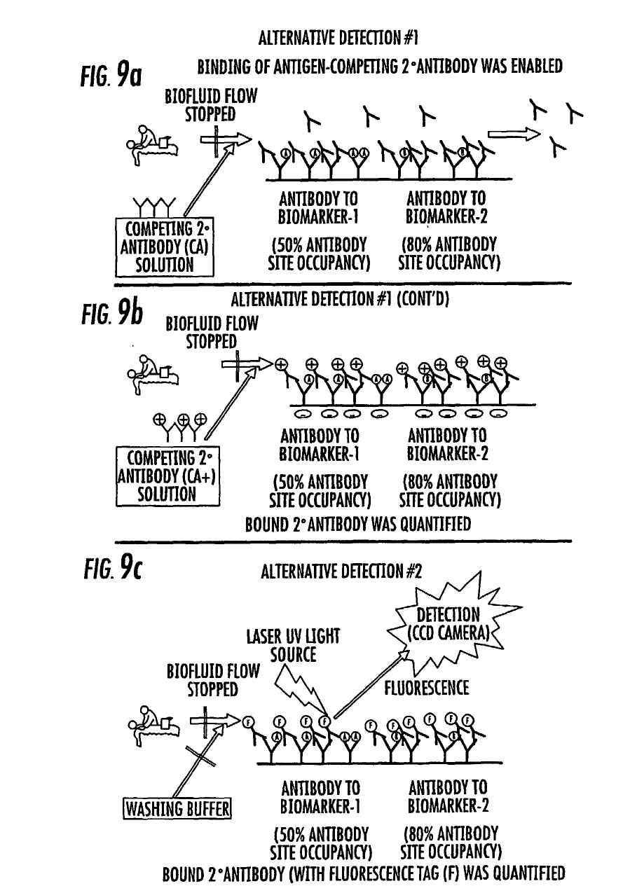

In another preferred embodiment, between the washing step and the biosensor regeneration step, the binding or occupancy frequency or levels are converted to an electrical signal or photo/light signal. A schematic illustration of the assay format is shown in FIG. 9A. For example, use of a competing detecting agent such as a competing antibody, against the immobilized detecting antibody. Selection is based on agents that directly or indirectly compete for binding with biomarker binding sites, that is, competing antibody will only bind free immobilized detecting antibody, but not biomarker-bound antibody (FIG. 9A). Modifications to the assay include diversion of the patient sample away from the biosensor during the time that the competing antibody solution is being introduced.

In another preferred embodiment, binding of the competing antibody is converted to an electrical signal by introducing surface charge to the crystal (negative) while positively charging the competing antibody (CA)⁺. See for example, FIG. 9B.

In another preferred embodiment, various fluorophores for multiple biomarker detection can be used. Different fluorophores are covalently linked to the different competing antibodies. Once bound, and after washing to remove unbound competing antibodies, lasers are used to excite the fluorophore (e.g. fluorescein, Texas Red and the Like at different wavelengths and fluorescein emission can then be detected at various wavelengths and converted to electric signal via photo multiplier tube. See for example, FIG. 9C.

In the embodiment, the biosensor is a quartz crystal microbalance (QCM) sensor, but can be another type of piezoelectric acoustic wave devices, including surface acoustic wave (SAW) devices, acoustic plate mode (APM) devices, and flexural plate wave (FPW) devices. Alternatively, fiber optic sensors and electrochemical sensors may be used.

Any equivalent measuring systems can be used to determine the binding of molecules to determine the detection of biomarkers at a given time in a sample.

In one embodiment of the invention, the binding partners of the plurality of protein-capture agents on the biosensor surface are proteins which are all expression products, or fragments thereof, of a cell or population of cells of a single organism. The expression products may be proteins, including peptides, of any size or function. They may be intracellular proteins or extracellular proteins. The expression products may be from a one-celled or multicellular organism. The organism may be a plant or an animal. In a preferred embodiment of the invention, the binding partners are human expression products, or fragments thereof.

In one embodiment of the invention, the binding partners of the protein-capture agents of the array may be a randomly chosen subset of all the proteins, including peptides, which are expressed by a cell or population of cells in a given organism or a subset of all the fragments of those proteins. Thus, the binding partners of the protein-capture agents of the sensor optionally represent a wide distribution of different proteins from a single organism.

The binding partners of some or all of the protein-capture agents on the biosensor need not necessarily be known. The binding partner of a protein-capture agent of the sensor may be a protein or peptide of unknown function. For instance, the different protein-capture agents of the sensor may together bind a wide range of cellular proteins from a single cell type, many of which are of unknown identity and/or function.

In another embodiment of the present invention, the binding partners of the protein-capture agents on the surface are related proteins. The different proteins bound by the protein-capture agents may optionally be members of the same protein family. The different binding partners of the protein-capture agents of the sensor may be either functionally related or just suspected of being functionally related. The different proteins bound by the protein-capture agents of the sensor may also be proteins which share a similarity in structure or sequence or are simply suspected of sharing a similarity in structure or sequence. For instance, the binding partners of the protein-capture agents on the array may optionally all be growth factor receptors, hormone receptors, neurotransmitter receptors, catecholamine receptors, amino acid derivative receptors, cytokine receptors, extracellular matrix receptors, antibodies, lectins, cytokines, serpins, proteases, kinases, phosphatases, ras-like GTPases, hydrolases, steroid hormone receptors, transcription factors, heat-shock transcription factors, DNA-binding proteins, zinc-finger proteins, leucine-zipper proteins, homeodomain proteins, intracellular signal transduction modulators and effectors, apoptosis-related factors, DNA synthesis factors DNA repair factors, DNA recombination factors, or cell-surface antigens.

In an alternative embodiment of the invention, the proteins which are the binding partners of the protein-capture agents of the sensor may be fragments of the expression products of a cell or population of cells in an organism.

A protein-capture agent on the sensor can be any molecule or complex of molecules which has the ability to bind a protein and immobilize it to the site of the protein-capture agent on the sensor. Preferably, the protein-capture agent binds its binding partner in a substantially specific manner. Hence, the protein-capture agent may optionally be a protein whose natural function in a cell is to specifically bind another protein, such as an antibody or a receptor. Alternatively, the protein-capture agent may instead be a partially or wholly synthetic or recombinant protein which specifically binds a protein, e.g. see Table 1. Alternatively, the protein-capture agent may be a protein which has been selected in vitro from a mutagenized, randomized, or completely random and synthetic library by its binding affinity to a specific protein or peptide target. The selection method used may optionally have been a display method such as ribosome display or phage display (see below). Alternatively, the protein-capture agent obtained via in vitro selection may be a DNA or RNA aptamer which specifically binds a protein target (for example: Potyrailo et al., Anal. Chem., 70:3419-25, 1998; Cohen, et al., Proc. Natl. Acad. Sci. USA, 95:14272-7, 1998; Fukuda, et al., Nucleic Acids Symp. Ser., (37):237-8, 1997). Alternatively, the in vitro selected protein-capture agent may be a polypeptide (Roberts and Szostak, Proc. Natl. Acad. Sci. USA, 94:12297-302, 1997). In an alternative embodiment, the protein-capture agent may be a small molecule which has been selected from a combinatorial chemistry library or is isolated from an organism.

In a preferred embodiment of the sensor, however, the protein-capture agents are proteins. In a particularly preferred embodiment, the protein-capture agents are antibodies or antibody fragments. Although antibody moieties are exemplified herein, it is understood that the present sensors and methods may be advantageously employed with other protein-capture agents.

The antibodies or antibody fragments of the sensor may optionally be single-chain Fvs, Fab fragments, Fab′ fragments, F(ab′)₂ fragments, Fy fragments, dsFvs diabodies, Fd fragments, full-length, antigen-specific polyclonal antibodies, or full-length monoclonal antibodies. In a preferred embodiment, the protein-capture agents of the array are monoclonal antibodies, Fab fragments or single-chain Fvs.

The antibodies or antibody fragments may be monoclonal antibodies, even commercially available antibodies, against known, well-characterized proteins. Alternatively, the antibody fragments have been derived by selection from a library using the phage display method. If the antibody fragments are derived individually by selection based on binding affinity to known proteins, then, the binding partners of the antibody fragments are known. In an alternative embodiment of the invention, the antibody fragments have been derived by a phage display method comprising selection based on binding affinity to the (typically, immobilized) proteins of a cellular extract or a body fluid. In this embodiment, some or many of the antibody fragments of the sensor would bind proteins of unknown identity and/or function.

The substrate of the sensor may be either organic or inorganic, biological or non-biological, or any combination of these materials. In one embodiment, the substrate is transparent or translucent. The portion of the surface of the substrate on which the patches reside is preferably flat and firm or semi-firm. However, the sensor of the present invention need not necessarily be flat or entirely two-dimensional. Significant topological features may be present on the surface of the substrate surrounding the patches, between the patches or beneath the patches. For instance, walls or other barriers may separate the patches of the sensor.

Numerous materials are suitable for use as a substrate in the invention. For instance, the substrate of the invention array can comprise a material such as, for example: silicon, silica, quartz, glass, controlled pore glass, carbon, alumina, titania, tantalum oxide, germanium, silicon nitride, zeolites, and gallium arsenide. Many metals such as gold, platinum, aluminum, copper, titanium, and their alloys are also options for substrates of the array. In addition, many ceramics and polymers may also be used as substrates. Polymers which may be used as substrates include, but are not limited to, the following: polystyrene; poly(tetra)fluoroethylene (PTFE); polyvinylidenedifluoride; polycarbonate; polymethylmethacrylate; polyvinylethylene; polyethyleneimine; poly(etherether)ketone; polyoxymethylene (POM); polyvinylphenol; polylactides; polymethacrylimide (PMI); polyalkenesulfone (PAS); polypropylethylene, polyethylene; polyhydroxyethylmethacrylate (HEMA); polydimethylsiloxane; polyacrylamide; polyimide; and block-copolymers.

A sensor of the present invention may optionally further comprise a coating between the substrate and the organic thinfilm of its patches. This coating may either be formed on the substrate or applied to the substrate. The substrate can be modified with a coating by using thin-film technology based, for instance, on physical vapor deposition (PVD), plasma-enhanced chemical vapor deposition (PECVD), or thermal processing. Alternatively, plasma exposure can be used to directly activate or alter the substrate and create a coating. For instance, plasma etch procedures can be used to oxidize a polymeric surface (for example, polystyrene or polyethylene to expose polar functionalities such as hydroxyls, carboxylic acids, aldehydes and the like) which then acts as a coating.

The coating is optionally a metal film. Possible metal films include gold, chromium, copper, molybdenum, nickel, palladium, silicon, zinc aluminum, titanium, tantalum, nickel, stainless steel, zinc, lead, iron, magnesium, manganese, cadmium, tungsten, cobalt, and alloys or oxides thereof. In a preferred embodiment, the metal film is a noble metal film. Noble metals that may be used for a coating include, but are not limited to, gold, platinum, silver, and copper. In an especially preferred embodiment, the coating comprises gold or a gold alloy. Electron-beam evaporation may be used to provide a tin coating of gold on the surface of the substrate. In a preferred embodiment, the metal film is from about 1 nm to about 1000 nm in thickness.

In alternative embodiments, the coating consists of a composition selected from the group consisting of silicon, silicon oxide, titania, tantalum oxide, silicon nitride, silicon hydride, indium tin oxide, magnesium oxide, alumina, glass, hydroxylated surfaces, and polymers.

Deposition or formation of the coating (if present) on the substrate is performed prior to the formation of the organic thinfilm thereon. Several different types of coating may be combined on the surface. The coating may cover the whole surface of the substrate or only parts of it. The pattern of the coating may or may not be identical to the pattern of organic thinfilm used to immobilize the protein-capture agents. In one embodiment of the invention, the coating covers the substrate surface only at the site of the patches of protein-capture agents. Techniques useful for the formation of coated patches on the surface of the substrate which are organic thinfilm compatible are well known to those of ordinary skill in the art. For instance, the patches of coatings on the substrate may optionally be fabricated by photolithography, micromolding (PCT Publication WO 96/29629), wet chemical or dry etching, or any combination of these.

The organic thinfilm on which each of the patches of protein-capture agents resides forms a layer either on the substrate itself or on a coating covering the substrate. The organic thinfilm on which the protein-capture agents of the patches are immobilized is preferably less than about 20 nm thick. In some embodiments of the invention, the organic thinfilm of each of the patches may be less than about 10 nm thick.

A variety of different organic thinfilm are suitable for use in the present invention. Methods for the formation of organic thinfilms include in situ growth from the surface, deposition by physisorption, spin-coating, chemisorption, self-assembly, or plasma-initiated polymerization from gas phase. For instance, a hydrogel composed of a material such as dextran can serve as a suitable organic thinfilm. In one preferred embodiment of the invention, the organic thinfilm is a lipid bilayer. In another preferred embodiment, the organic thinfilm is a monolayer. A monolayer of polyarginine or polylysine adsorbed on a negatively charged substrate or coating is one option for the organic thinfilm. Another option is a disordered monolayer of tethered polymer chains. In an embodiment the organic thinfilm is a self-assembled monolayer. The organic thinfilm is most preferably a self-assembled monolayer which comprises molecules of the formula X—R—Y, wherein R is a spacer, X is a functional group that binds R to the surface, and Y is a functional group for binding protein-capture agents onto the monolayer. In an alternative embodiment, the organic thinfilm comprises a combination of organic thinfilm such as a combination of a lipid bilayer immobilized on top of a self-assembled monolayer of molecules of the formula X—R—Y. As another example, a monolayer of polylysine can also optionally be combined with a self-assembled monolayer of molecules of the formula X—R—Y (see U.S. Pat. No. 5,629,213).

A variety of techniques may be used to generate patches of organic thinfilm on the surface of the substrate or on the surface of a coating on the substrate. These techniques are well known to those skilled in the art and will vary depending upon the nature of the organic thinfilm, the substrate, and the coating if present. The techniques will also vary depending on the structure of the underlying substrate and the pattern of any coating a present on the substrate. For instance, patches of a coating which is highly reactive with an organic thinfilm may have already been produced on the substrate surface. Arrays of patches of organic thinfilm can optionally be created by microfluidics printing, microstamping (U.S. Pat. Nos. 5,512,131 and 5,731,152), or microcontact printing (μCP) (PCT Publication WO 96/29629). Inkjet printer heads provide another option for patterning monolayer X—R—Y molecules, or components thereof, or other organic thinfilm components to nanometer or micrometer scale sites on the surface of the substrate or coating (Lemmo et al., Anal Chem., 1997, 69:543-551; U.S. Pat. Nos. 5,843,767 and 5,837,860). In some cases, commercially available arrayers based on capillary dispensing (for instance, OmniGrid™ from Genemachines, Inc, San Carlos, Calif., and High-Throughput Microarrayer from Intelligent Bio-Instruments, Cambridge, Mass.) may also be of use in directing components of organic thinfilms to spatially distinct regions of the sensor.

Diffusion boundaries between the patches of protein-capture agents immobilized on organic thinfilms such as self-assembled monolayers maybe integrated as topographic patterns (physical barriers) or surface functionalities with orthogonal wetting behavior (chemical barriers). For instance, walls of substrate material or photoresist may be used to separate some of the patches from some of the others or all of the patches from each other. Alternatively, non-bioreactive organic thinfilms, such as monolayers, with different wettability may be used to separate patches from one another.

Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated in pertinent part, by reference for all purposes to the same extent as if each individual publication or patent document were so individually denoted. By their citation of various references in this document, Applicants do not admit any particular reference is “prior art” to their invention.

### EXAMPLES

The following examples serve to illustrate the invention without limiting it thereby. It will be understood that variations and modifications can be made without departing from the spirit and scope of the invention.

### Example 1

### Real-Time Monitoring of Samples

A prototype biomonitor device was assembled as illustrated (FIG. 10). The device entails use of commercially available (International Crystal Manufacturing Company, Inc.) AT cut quartz crystal oscillator with a 100 nm thick, 0.5 mm diameter gold surface (FIG. 11). The quartz crystal is made to operate by attachment to an RF lever oscillator drive circuit that has a nominal drive frequency of 10 MHz and is commercially available from the same company. The quartz crystal is sandwiched between two rubber o-rings within a 75 μL liquid flow cell (FIG. 11) that permits contact of a fluid stream with the gold surface. The quartz crystal, flow cell, and oscillator circuitry are encapsulated within a styrofoam enclosure to reduce signal noise by minimizing temperature fluctuation. An electronically controlled six-port Rheodyne flow valve is used to deliver a user selected buffer or sample to the flow cell. A Harvard Apparatus syringe pump was used to provide a flowrate of 50 μL/min. The quartz crystal resonating frequency and amplitude are monitored by use of an Agilent frequency counter and a National Instruments analog-to-digital converter, respectively. The change from initial oscillation frequency and amplitude are recorded and displayed using custom software to derive change in surface adsorption.

A 1 mg/mL solution of protein A in phosphate buffered saline (PBS) was used to test the response of the prototype biomonitor under flow conditions. The gold surface of the quartz crystal was pre-treated with piranha solution for five minutes and dried with nitrogen gas. After the quartz crystal was inserted into the flow cell, the system was equilibrated with PBS for 5 minutes at 50 μL/min. Data acquisition is begun while continuing the PBS flow, at which point the initial oscillation frequency and amplitude are set. After one minute, the flow selection valve is switched to pass the protein A solution through the flow cell at 50 μL/min (FIG. 11). A change in frequency reflected the adsorption of protein A onto the gold surface, forming a monolayer within approximately one minute. The adsorption kinetics then changed to reflect the formation of secondary layers atop of the initial monolayer. After 20 minutes, the fluid stream was switched back to PBS in order to wash the surface of weakly bound protein A and contaminants. An increased frequency reflected removal of the secondary molecular layers, leaving behind the gold-adhered monolayer. The final frequency change of −30 Hz reflects the adsorption of the protein A monolayer.

Atomic force microscopy (AFM) images were collected to characterize the immobilization of an antibody onto a 100 nm thick gold surface via Protein A linkage (FIG. 13). The gold surface of a quartz crystal was pre-treated with piranha solution for five minutes and dried with nitrogen gas. An AFM image of the clean surface was captured (FIG. 13 at right). Next, 100 μL of a protein A solution (1 mg/mL in Tris-HCl pH 7.4) was applied to the surface for one hour, and washed 3 times with PBS; a second AFM image of the protein A coated gold was captured. Finally, 100 μL of a 0.25 mg/mL anti-streptavidin-ALP solution was added to the protein A coated gold surface for one hour and washed three times in PBS. The final AFM image was captured showing the significant increase in density of the antibody—protein A is known to bind up to four antibodies per molecule. AFM images were captured on a Digital Instruments Dimension 3100 operated in contact mode over a 10×10 μm region of the gold surface.

Antibody immobilization: A 1 mg/mL protein A in PBS solution (100 μL) was incubated atop the 100 nm thick gold surface for one hour. Following the incubation, the surface was washed 3 times (10 minutes each) with PBS. Atop the protein A coated gold was placed 100 μL of a 0.25 mg/mL anti-streptavidin solution in Tris-hydrochloride buffer (pH 7.4). After one hour of incubation, the surface was washed three times with PBS (10 minutes each). The stability of the antibody-protein A complex was strengthened by crosslinking the two using a 30 mM solution of 1,5 dimethylpimelimidate dihydrochloride in a triethanol amine buffer (pH 8.5). Non-crosslinked components were washed from the surface by washing with a glycine hydrochloride buffer (pH 2.3) for 3 minutes. Afterward the surface was rinsed 3 times for ten minutes with PBS.

Antigen incubation time: In an experiment conducted in triplicate, immobilized antibody treated gold surfaces were incubated with 0.025 mg/mL solutions of streptavidin-alkalinephosphatase (antigen) in Tris-hydrochloride buffer (pH 7.4) for 1, 2, 3, 5, 7, 10, 13, 15, 17, 20, 23, 27, and 30 minutes. The relative quantity of antigen binding was recorded by reacting the surface with a 125 μL solution of 1 mg/mL para-nitrophenylphosphate (PNPP) (pH 10). The alkalinephosphatase (ALP) hydrolyzed the pNPP within 12.5 minutes after which the solution was aspirated and mixed with 25 μL of 3M NaOH to terminate the reaction. The absorbance of the final solution was recorded at 405 nm and plotted for each antigen incubation time in FIG. 14. The time:antigen binding kinetics illustrated in FIG. 14 suggest that an endpoint of 90% binding is reached within approximately 10 minutes. Though the exponential nature of the kinetics suggests that this is a second order reaction, and thus is susceptible to the concentration of both the antigen and the antibody.

Calibration curve: In a set of three replicate experiments, 100 μL of six streptavidin-ALP (antigen) solutions at concentrations of 0, 0.00125, 0.0025, 0.005, 0.010, 0.025 mg/mL in Tris-hydrochloride buffer (pH 7.4) were placed atop immobilized antibody treated gold surfaces for 10 minutes (the previous optimized time). Following antigen binding, the surface was washed three times with PBS. The ALP conjugate was again used to hydrolyze pNPP as described above. The solution absorbance was plotted against antigen concentration in FIG. 15, which demonstrated a linear response of the immobilized antigen surface within the tested concentration range.

Nonspecific interactions: Two experiments (n=3) were conducted to assess nonspecific binding with the treated gold surface. In the first, the gold surface was coated with protein A and in the second protein A coated gold was treated with anti-actin antibody as a non-specific compliment to the streptavidin-ALP antigen. In the first experiment, the gold surfaces were treated with protein A as described earlier, incubated with streptavidin-ALP (0.025 mg/ml, 100 μl) for 10 minutes, and reacted with pNPP for 12.5 minutes with the absorbance read at 405 nm. The plates were then stored at 4° C. overnight for 4 days with the antigen binding procedure repeated each day. The average absorbance reading for each day remained constant at 0.08 absorbance units. In the second experiment (n=7), the gold surface was treated with protein A as described earlier then incubated with 100 μL solution of anti-actin (0.75 mg/mL in Tris-hydrochloride buffer). After the incubation with the antibody, the plates were incubated with crosslinker and washed as described earlier. After incubation with streptavidin-ALP for 10 minutes and reaction with pNPP for 12.5 minutes, and the absorbance was read. The reading was 0.09 absorbance units. Together these experiments indicated a non-specific absorbance reading of <0.1 absorbance units using the pNPP detection procedure, which remained constant over 4 days of treatment.

Plate regeneration: Four separate experiments were conducted to evaluate the short term and long term regeneration performance of the immobilized antibody surface at room temperature and 4° C. The gold surface was treated with protein A, anti-streptavidin, and crosslinker as described earlier. Gold surfaces (n=3) were then incubated with streptavidin-ALP at room temperature and 4° C., and reacted with pNPP for absorbance measurement. Afterwards, chips were treated with glycine-hydrochloride buffer (pH 2.3) for 3 minutes and washed with PBS. In the short term experiments, re-incubated with streptavidin-ALP and pNPP was performed in increments of minutes and hours (FIGS. 16 and 17). In the long term experiments, re-incubation with streptavidin-ALP and pNPP was performed in increments of days (FIGS. 18 and 19). The results show that regeneration is possible for three to four cycles before the absorbance approaches that of non-specific interactions. Each cycle shows a drop in absorbance for the same antigen concentration, whether at long term, short term, room temperature or 4° C. conditions. These data suggest that the immobilized antibody—protein A complex is not sufficiently stabile for regeneration by glycine-hydrochloride treatment.

New immobilization techniques: Based on the non-covalent nature of protein A binding with the gold surface, it is expected that this is the weakest point in the antibody immobilization process that prevents effective repeated measurements. We suggest three additional strategies to improve antibody immobilization to the gold surface.

Based on work reported by Ren et al. (Analyst 2000, v125:669-671), a cysteine tag can be added recombinantly to the F_{c} portion of the protein A molecule. This will provide a covalent linkage between gold and protein A that should improve the stability of the immobilized antibody complex.

The gold surface can be treated with biotin and coated with streptavidin as suggested by Peluso et al. (Analytical Biochemistry 2003, v312:113-124). The biomarker antibody can then be biotinylated on its F_{c} portion and bound to the streptavidin coated gold surface without the use of protein A.

As also described by Peluso et al. in the same manuscript, an antibody can be disassembled to remove the F_{c} portion from the F_{ab} portion. Then the disulfide bond between the two halves of the F_{ab} portion can be reduced to leave free thiol groups. This procedure can be performed with the biomarker antibody. Different from that presented by Peluso et al. is the concept that this newly formed thiol groups at the end of the Fₐ and F_{b} antibody portions, which is opposed to the binding end of the Fₐ and F_{b} antibody portions, can be directly covalently linked to the gold surface. This Novel suggestion is likely to present a simple, high density, and robust means to complex the binding domains of biomarker antibodies with a gold surface.

The software has been written in-house using the LabView programming environment. The software controls actuation and tuning of the fluid selection valve. The software interfaces with the frequency counters to collect the oscillation frequency. The initial frequency at the beginning of acquisition is recorded, with the difference in frequency calculated in respect to the initial value. The frequency difference is plotted against time and recorded to a data file. The software also interfaces with the National Instruments analog-to-digital converter and records the oscillation amplitude output by the RF oscillator circuit. The initial amplitude at the beginning of acquisition is recorded, with the difference in amplitude calculated with respect to the initial value. The amplitude difference is plotted against time and recorded to a data file.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A system for detecting and diagnosing neural injury in a patient, said system comprising:
a biosensor;
a flow cell for delivery of a sample from said patient to said biosensor;
a capture probe adsorbed to surface of said biosensor; and,
a computational system communicably connected to said biosensor, said computational system correlating the detection of one or more protein biomarkers in said sample with a diagnosis of neural injury and/or neuronal disorders, wherein the correlation takes into account the detection of one or more protein biomarkers in each diagnosis, as compared to normal subjects.

2. The system of claim 1, wherein the biosensor is a quartz crystal microbalance which comprises a surface adsorbed capture molecule.

3. The system of claim 1, wherein the surface adsorbed capture molecule is an antibody.

4. The system of claim 3, wherein the antibody specifically binds neural biomarkers, peptides, fragments, variants or derivatives thereof.

5. The system of claim 1, wherein the biosensor is subjected to an equilibration step by a flow through of renewing buffer.

6. The system of claim 1, wherein the renewing buffer removes unbound capture molecule.

7. The system of claim 1, wherein the system further comprises a patient sample flow regulator.

8. The system of claim 1, wherein a patient sample flows from a patient into a flow cell.

9. The system of claim 8, wherein the flow cell regulates the flow rate of sample and buffers.

10. The system of claim 1, wherein the system further comprises a reference biosensor.

11. The system of claim 1, wherein binding of the capture molecule, adsorbed to the surface of the quartz crystal microbalance, to a ligand produces a detectable resonance.

12. The system of claim 1, wherein the resonance is indicative of specific binding.

13. The system of claim 12, wherein the resonance is about 1 Hz.

14. The system of claim 12, wherein the resonance is about 5 Hz.

15. The system of claim 12, wherein the resonance is about 10 Hz.

16. The system of claim 12, wherein the resonance is up to about 40 Hz.

17. The system of claim 1, wherein at least one biomarker is detected.

18. The system of claim 1, wherein a plurality of biomarkers are detected.

19. The system of claim 1, wherein capture molecules of different specificities are adsorbed to addressable locations on the surface of biosensor.

20. A method for detection and identification of protein biomarkers in a patient comprising:
providing a patient sample;
capturing of biomarkers on a substrate surface by a surface substrate bound capturing molecule;
applying an oscillating electric field across the substrate surface;
measuring at least one resonant frequency of the substrate surface;
measuring the admittance magnitude at the resonant frequencies simultaneously, and correlating the resonant frequency and the admittance magnitude to obtain a surface mass density; thereby,
detecting and identifying one or more biomarkers.

21. The method of claim 20, wherein a computational system correlates surface mass density with the amount of biomarker bound by the capture molecule.

22. The method of claim 20, wherein the substrate surface is subjected to an equilibration step which removes any unbound molecules.

23. The method of claim 20, wherein the substrate surface is in contact with a buffer which optimizes binding reactions between the biomarker and capture probe.

24. The method of claim 20, wherein the substrate surface comprising the bound biomarker capture molecule is washed with disassociation buffer to remove biomarkers bound by the capture molecule.

25. The method of claim 24, wherein the disassociation buffer disassociates the biomarker from the capture molecule without removing the capture molecule from the surface of the surface substrate.

26. The method of claim 20, wherein the substrate surface is washed with washing buffer to allow binding of biomarkers from a successive sample from a patient.

27. The method of claim 20, wherein the substrate surface is reused in successive measurements of biomarkers in a sample.

28. A method of real-time measurement of biomarkers in a patient sample comprising the steps of equilibration; antigen association; antigen disassociation and regeneration.

29. The method of claim 28, wherein the equilibration step is performed after a capture molecule is adsorbed to a biosensor surface.

30. The method of claim 28, wherein the sample from a patient flows at a rate of 1 ml per 10 minutes over the surface of the biosensor allowing for binding of ligand and antibody.

31. The method of claim 28, wherein the sample from a patient flows at a rate of 1 ml per 30 minutes over the surface of a biosensor allowing for binding of ligand and capture molecule.

32. The method of claim 28, wherein the sample comprises biomarkers diagnostic of neural injury.

33. The method of claim 28, wherein the biosensor detects at least one biomarker.

34. The method of claim 28, wherein the biosensor detects about two biomarkers.

35. The method of claim 28, wherein the biosensor detects about five biomarkers.

36. The method of claim 28, wherein the biosensor detects the biomarkers in a patient sample and data generated from the detection is acquired on a portable computer.

37. The method of claim 36, wherein the data provides a real-time monitoring of a patient suffering from neural injury.

38. The method of claim 37, wherein the data is generated within about 5 minutes of the patient sample flow-through.

39. The method of claim 37, wherein the data is generated within about 2 minutes of patient sample flow through.

40. The method of claim 37, wherein the data is generated within about 1 minute of patient sample flow through.

41. The method of claim 37, wherein the data is generated within about thirty seconds of patient sample flow through.

42. The method of claim 28, wherein a successive patient sample comprises biomarkers that bind to capture molecules after the biosensor is washed with disassociation buffer and releasing previously bound biomarkers.

43. The method of claim 42, wherein binding of different biomarkers is diagnostic of type of neural injury, the location of the injury, and the degree of severity of the injury.

44. The method of claim 28, wherein at least one biomarker is indicative of a type of neural injury.

45. The method of claim 28, wherein a plurality of biomarkers are indicative of the type of neural injury.

46. The method of claim 28, wherein at least one biomarker is indicative of the location of neural injury.

47. The method of claim 28, wherein a plurality of biomarkers is indicative of the in vivo location of neural injury.

48. The method of claim 28, wherein at least one biomarker is indicative of the degree of severity of neural injury.

49. The method of claim 28, wherein a plurality of biomarkers are indicative of the degree of severity of neural injury.

50. The method of claim 28, wherein the antibody specifically binds neural biomarkers, peptides, fragments, variants or derivatives thereof.

51. A system to monitor patient samples in real time comprising:
a quartz crystal oscillator attached to an RF lever oscillator drive circuit sandwiched between a water-tight compression fitting within a liquid flow cell and encapsulated within an enclosure to reduce signal noise by minimizing temperature fluctuation; and,
an electronically controlled six-port flow valve;
a frequency counter;
an analog to digital converter; and,
software to analyze data.

52. The system of claim 51, wherein the quartz crystal is about 1 nm up to 1000 nm thick and has a diameter of between about 0.1 mm up to 25 mm.

53. The system of claim 51, wherein the quartz crystal is an AT, BT or AZ cut quartz crystal.

54. The system of claim 51, wherein the RF lever oscillator drive circuit has a drive frequency of at least about 10 MHz.

55. The system of claim 51, wherein the water-tight compression fitting are the two rubber O-rings.

56. The system of claim 51, wherein the liquid flow cell and encapsulated is within styrofoam.
